(19) United States
(12) Patent Application Publication
(10) Pub. No.: US 2018/0113119 A1
(43) Pub. Date: Apr. 26, 2018

(54) METHODS FOR CHARACTERIZING TIME-BASED HEPATOTOXICITY

(71) Applicant: HUREL CORPORATION, Beverly Hills, CA (US)

(72) Inventors: Robert FREEDMAN, Beverly Hills, CA (US); James MACDONALD, Chester, NJ (US); Eric NOVIK, Edison, NJ (US)

(73) Assignee: HUREL CORPORATION, Beverly Hills, CA (US)

(21) Appl. No.: 15/559,452

(22) PCT Filed: Mar. 21, 2016
(86) PCT No.: PCT/US16/23477

§ 371 Date: Sep. 19, 2017

Related U.S. Application Data
(60) Provisional application No. 62/136,428, filed on Mar. 20, 2015.

(51) Int. Cl. G01N 33/50

(52) U.S. Cl. CPC G01N 33/5067; G01N 33/5014

(57) ABSTRACT

Methods of characterizing the time-based hepatotoxicity of a test compound are provided. In some embodiments the provided methods allow prediction of the hepatotoxic potential of a test compound in a way that is improved relative to that achievable with prior art methods. In some embodiments the methods may be used to quantify the relationship between measurements or estimates of toxicity made at different, successive points in time, to provide highly meaningful tool to assess the likely in vivo hepatotoxicity of test compounds in vivo.

A major obstacle in the development of new drugs is taking drug candidates forward that will exhibit both efficacy and safety sufficient to attain regulatory approval. Often a promising candidate will fail at the preclinical or clinical stage. While there are many reasons for drug candidate attrition in the development process, one frequent reason is safety-related findings related to potential liver toxicity. There is a need in the art to significantly reduce costs resulting from clinical and late-stage pre-clinical failures due to findings of liver toxicity, to reduce the time to identification of safety-related issues, and to speed decision-making on candidate compounds identified by basic discovery research.

The battery of in vitro studies currently employed to support selection of candidates with the highest probability of success is deficient in its ability to detect hepatotoxicity. Hepatotoxicity may result from one or more mechanisms including genetic toxicity (mutagenesis, clastogenesis), CYP inhibition (including time dependent inhibition), CYP induction, G-SH adduct formation, covalent binding to protein, and hERG current inhibition, steatosis, granuloma, formation of ractive metabolites, and cholestasis. In vitro cytotoxicity assessment with conventional hepatocyte systems—including in vitro cell-based models that afford short-term or acute testing, often with incubations that yield data only at short-term time points of around 24 hours or less, or only at one such time point—does not come even close to adequately predicting in vivo hepatotoxic potential. Indeed, many practicing toxicologists skilled in the relevant art consider data derived from such short-term, “acute” in vitro tests to have little or no actionable value in pre-clinical decision-making, and they frequently continue to progress drug compounds into subsequent (and increasingly expensive) stages of development despite the positive findings of hepatotoxic response (sometimes referred to in the art as positive “liver signals”) that these models may generate. Accordingly, there is a need in the art for new and improved methods and systems of characterizing hepatotoxicity. This invention meets these and other needs.

### SUMMARY

One reason for the limitations of current in vitro systems is they are generally limited to a twenty-four hour readout because of limited and/or diminishing metabolic competency. This invention utilizes hepatocyte cultures capable of maintaining in vitro functionality for an extended period of time of up to, for example 14, 21, or 28 days in culture. Based in part on the use of such systems this invention provides methods comprising measuring hepatotoxicity in culture at a plurality of time points over a period of one, two, three, four, five, six, seven or more, or fourteen days or longer. In some embodiments the provided methods allow prediction of the hepatotoxic potential of a test compound in a way that is improved relative to that achievable with prior art methods. In some embodiments the methods may be used to quantify the relationship between measurements or estimates of toxicity made at different, successive points in time. By comparing toxicity of test compounds measured at 24 h with toxicity measured at seven days or at 14 days, for example, it has been discovered that changes in toxicity over time provide highly meaningful tool to assess the likely in vivo hepatotoxicity of test compounds in vivo.

Accordingly, in some embodiments this invention provides methods of characterizing the time-based hepatotoxicity of a test compound. In some embodiments the methods comprise a) incubating a first in vitro culture comprising hepatocytes with a test compound for a first culture period; b) measuring at least one cytotoxic effect of the test compound on the hepatocytes of the first in vitro culture over the first culture period to thereby define the hepatotoxicity of the test compound over the first culture period; c) incubating a second in vitro culture comprising hepatocytes with the test compound for a second culture period that is longer than the first culture period; d) measuring at least one cytotoxic effect of the test compound on the hepatocytes of the second in vitro culture over the second culture period to thereby define the hepatotoxicity of the test compound over the second culture period; and e) comparing the hepatotoxicity of the test compound over the first culture period to the hepatotoxicity of the test compound over the second culture period to thereby characterize the time-based hepatotoxicity of the test compound. In some embodiments the hepatotoxicity of the test compound over the second culture period is greater than the hepatotoxicity of the test compound over the first culture period and the test compound is identified as exhibiting time-based hepatotoxicity. In some embodiments the hepatotoxicity of the test compound over the second culture period is greater than the hepatotoxicity of the test compound over the first culture period by at least a pre-defined threshold and the test compound is identified as exhibiting time-based hepatotoxicity. In some embodiments the hepatotoxicity of the test compound over the second culture period is not greater than the hepatotoxicity of the test compound over the first culture period by at least a pre-defined threshold and the test compound is identified as not exhibiting time-based hepatotoxicity. In some embodiments the pre-defined threshold is selected from about 2, about 3, about 4, about 5, about 6, about 7, about 8, about 9, or about 10. In some embodiments the pre-defined threshold is selected from 2, 3, 4, 5, 6, 7, 8, 9, or 10. In some embodiments the pre-defined threshold is from 2 to 5, from 3 to 5, from 2 to 4, from 2 to 6, from 4 to 8 or from 5 to 10.

In some embodiments the first and second in vitro cultures comprise isolated hepatocytes. In some embodiments the isolated hepatocytes are substantially dispersed across the surface of a solid substrate. In some embodiments the hepatocytes are primary hepatocytes. In some embodiments the hepatocytes are stem cell-derived.

In some embodiments the first and second in vitro cultures further comprise at least one defined stromal cell type.

In some embodiments the first and second in vitro cultures comprise at least one of cultured hepatocytes configured in a micropattern comprising hepatocytes and at least one other cell type, and adhered to a substrate material; hepatocytes clustered in a spheroidal configuration of cells that is or is not encapsulated in a droplet of culture medium or adhered to microbeads; hepatocytes deposited in a “sandwich” culture wherein the hepatocytes are stabilized between basal and overlay layers of collagen or other gel or protein; and hepatocytes embedded in or on a membrane, lattice, scaffold, or bed of naturally occurring or man-made materials such as, without limitation, scaffold or lattice comprised of material derived from alginate, a man-made semi-permeable membrane, or a bed comprised of cultured blood capillaries.

In some embodiments the first culture period is from twelve hours to two days. In some embodiments the first culture period is one day. In some embodiments the second culture period is from three to twenty-eight days. In some embodiments the second culture period is from seven to fourteen days. In some embodiments the first culture period is one day and the second culture period is seven days or fourteen days.

In some embodiments a culture period of “x” days is a culture period of at least “x” days. In some embodiments a culture period of “x” days is a culture period of at least about “x” days. In some embodiments a culture period of “x” days is a culture period of about “x” days.

In some embodiments the method further comprises at least a third culture period, and a) incubating the third in vitro culture comprising hepatocytes with the test compound for a third culture period that is longer than the first and the second culture period; b) measuring at least one cytotoxic effect of the test compound on the hepatocytes of the third in vitro culture over the third culture period, to thereby define the hepatotoxicity of the test compound over the third culture period; and c) comparing the hepatotoxicity of the test compound over the first, the second, or both the first and the second culture period(s) to the hepatotoxicity of the test compound over the third culture period to thereby characterize the time-based hepatotoxicity of the test compound.

In some embodiments of the methods the comparing comprises performing a mathematical operation comprising determining the arithmetic ratio between two or more measured quantities, or determining the relationship between two measured quantities utilizing methods of integral or differential calculus, or utilizing probabilistic, stochastic, or simulative forms of measurement or computational models.

In some embodiments step a) comprises incubating a plurality of first in vitro cultures comprising hepatocytes are incubated with the test compound for the first culture period.

In some embodiments step c) comprises incubating a plurality of second in vitro cultures comprising hepatocytes with the test compound for the second culture period.

In some embodiments step b) comprises determining an TC₅₀ of the test compound over the first culture period.

In some embodiments step d) comprises determining an TC₅₀ of the test compound over the second culture period.

In some embodiments step b) comprises determining an TC₅₀ of the test compound over the first culture period, wherein step d) comprises determining an TC₅₀ of the test compound over the second culture period, and wherein step e) comprises determining the ratio of the TC₅₀ of the test compound over the first culture period to the TC₅₀ of the test compound over the second culture period to define a time based toxicity score for the test compound.

### BRIEF DESCRIPTION OF THE DRAWINGS

FIG. 1 shows that phase I enzyme function is long enduring in rat, dog, and primate hepatocyte-stromal cell cocultures of the invention. The rate of 7-hydroxycoumarin formation was measured at the indicated timepoints. Different patterns are evident for the different species; however, in each case phase-I enzyme function is long enduring. Rat and dog have phase I activity for at least 35 days and primate for at least 22 days from the day the hepatocytes are seeded.

FIG. 2 shows that phase II enzyme function is long enduring in rat, dog, and primate hepatocyte-stromal cell cocultures of the invention. The rate of 7-hydroxycoumaringlucuronide formation was measured at the indicated timepoints. Different patterns are evident for the different species; however, in each case phase-II enzyme function is long enduring. Rat and dog have phase II activity for at least 35 days and primate for at least 22 days from the day the hepatocytes are seeded.

FIG. 3 shows long enduring CYP 3A4 function in hepatocyte-stromal cell cocultures made from three different lots of primary human hepatocytes. CYP 3A4 function was analyzed by measuring the rate of 1-hydroxymidazolam formation. Some lot to lot variation in baseline was observed, but all lots have 3A4 activity for 35 days from the day the cells are seeded.

FIG. 4 shows long enduring CYP 2C9 function in hepatocyte-stromal cell cocultures made from three different lots of primary human hepatocytes. CYP 2C9 function was analyzed by measuring the rate of 4-hydroxytolbutamide formation. Some lot to lot variation in baseline was observed, but all lots have 2C9 activity for 35 days from the day the cells are seeded.

FIG. 5 shows staining of canaliculi in hepatocyte-stromal cell cocultures established using primary human, dog, rat, and primate hepatocytes.

FIG. 6 shows that hepatocyte CYP 3A4 activity is stable in human hepatocyte-stromal cell cocultures from days 7 to 25 days in culture. By day 7 the cultured primary hepatocytes have finished remodeling and stabilized function. Function remains stable through day 25. The window of stable function thus continues for 18 days. It is noteworthy that two weeks of stable culture is a long enough time for most DMPK and Tox applications.

FIG. 7 shows that four different CYP enzymes remain stable between days 7 and 18 of culture in most or all of three different lots of human hepatocytes in human hepatocyte-stromal cell cocultures.

FIG. 8 shows a multi species window of stability for phase I metabolism.

FIG. 9 shows a multi species window of stability for phase I metabolism.

FIG. 10 compares the HepG2 cell line to hepatocyte-stromal cell cocultures. Flutamide and ketoconazole are hepatotoxic. The data show that greater hepatic clearance of a toxic parent compound (flutamide or ketoconazole) requires a higher parent concentration to produce an equivalent level of toxicity. Troglitazone hepatotoxicity is mediated by its hepatotoxic metabolites. The primary reactive metabolite of troglitazone has been confirmed to be an o-quinone methide. This metabolite is generally formed by A GSH conjugation via oxidation of the substituted chromane ring system to a reactive o-quinone methide derivative. The data show that greater generation of toxic metabolite requires lower parent concentration to produce equivalent level of toxicity. Taken together these data demonstrate the superior predictive capabilities of the hepatocyte-stromal cell cocultures of the invention in comparison to HepG2 monocultures.

FIG. 11 shows the hepatotoxic effect of multiple dosing of cyclophosphamide on hepatocyte-stromal cell cocultures comprising human, dog, primate, or rat primary hepatocytes. Cell viability was analyzed using a CellTiter-Blue (Promega) assay, n=4. The data show that extending the duration of dosing increases the cyclophosphamide cytotoxicity.

FIG. 12 shows the hepatotoxic effect (calculated TC₅₀) of multiple dosing of troglitazone on hepatocyte-stromal cell cocultures comprising human, dog, primate, or rat primary hepatocytes following six days of treatment (three doses). The results in dog, monkey, and rat, are each individually compared to human in the three graphs presented in FIG. 12.

FIG. 13 summarizes the results for cyclophosphamide and troglitazone. These are concentration dependent toxicity curves generated after 6 days of repeat compound exposure in the 4 different species. Each compound was dosed every 2 days starting on day 0. On the 6^{th} day the Promega Celltiter blue assay was used to measure the conversion of resazurin to the fluorescent resorufin. Resazurin is effectively reduced in mitochondria so this assay measures mitochondrial metabolic activity. This data was then used to generate TC₅₀ values (i.e., the concentration of the compound at which fifty percent of the cells in the culture die in response to the dose administered) as follows. For Troglitazone: LC₅₀=308 μM (Human), LC₅₀=267 μM (Dog), LC₅₀=207 μM (Monkey), and LC₅₀=317 μM (Rat). For Cyclophosphamide: LC₅₀=5,523 μM (Human), LC₅₀=7,541 μM (Dog), LC₅₀=1,904 μM (Monkey), LC₅₀=1,506 μM (Rat).

FIG. 14 presents a comparison of the GSH and Promega Celltiter blue assays. The assay was done at 50*Cmax. The very low inter-assay variability is a further demonstration of the usefulness and capabilities of the systems and methods of this disclosure.

FIG. 15 lists twenty model hepatotoxicant compounds used in the examples. The third column indicates whether the compound is a known strong hepatotoxicant, an known moderate hepatotoxicant, or known to be free of hepatotoxicity. The second column indicates the probable or possible mechanism of hepatotoxicity induced by the known hepatotoxicants.

FIG. 16 presents observed hepatotoxicity for each of the twenty compounds in monocultures of human hepatocytes at 24 h, and in human hepatocyte-stromal cell co-cultures at 24 h, 7 d, and 14 d. The units of the TC50 values are micromolar.

FIG. 17 presents computations of time-based toxicity signals observed in human hepatocyte cocultures.

FIG. 18 presents observed hepatotoxicity for each of the twenty compounds in monocultures of rat hepatocytes at 24 h, and in rat hepatocyte-stromal cell co-cultures at 24 h, 7 d, and 14 d. The units of the TC50 values are micromolar.

FIG. 19 presents computations of time-based toxicity signals observed in rat hepatocyte cocultures.

FIGS. 20A, 20B, and 20C are parts of a single table and show that time-based toxicity signals are correlated to known mechanisms of toxicity of test compounds.

FIG. 21 shows that time-based toxicity signals for a set of ten compounds developed to a pre-clinical or clinical stage are correlated with observed toxicity.

FIG. 22 shows that time-based toxicity signals for a set of ten compounds developed to a pre-clinical or clinical stage are correlated with observed toxicity.

### DETAILED DESCRIPTION

Before the present systems and methods, and other embodiments are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

The term “comprising” as used herein is synonymous with “including” or “containing”, and is inclusive or open-ended and does not exclude additional, unrecited members, elements or method steps.

As used herein, “hepatotoxicity” may include any form of chemical driven liver damage, including without limitation liver damage caused by one or more mechanisms selected from genetic toxicity (mutagenesis, clastogenesis), CYP inhibition (including time dependent inhibition), CYP induction, G-SH adduct formation, covalent binding to protein, and hERG current inhibition, steatosis, granuloma, formation of ractive metabolites, and cholestasis.

### A. Hepatocyte Cultures

This disclosure provides systems and methods of assessing the toxicity of a test compound that comprise administering the test compound to cultured hepatocytes. In some embodiments the cultured hepatocytes are configured in a micropattern comprising hepatocytes and at least one other cell type, and adhered to a substrate material; clustered in a spheroidal configuration of cells that is or is not encapsulated in a droplet of culture medium or adhered to microbeads; deposited in a “sandwich” culture wherein the hepatocytes are stabilized between basal and overlay layers of collagen or other gel or protein; or embedded in or on a membrane, scaffold, or bed of naturally occurring or man-made materials such as, without limitation, scaffold or lattice comprised of material derived from alginate, a man-made semi-permeable membrane, or a bed comprised of cultured blood capillaries. In some embodiments the systems and methods are directed to assessing the toxicity of a test compound by administering it to cultured cells drawn from the heart, the liver, the brain, the central nervous system, muscle, bone or bone marrow, the lung, the gastrointestinal wall, the blood-brain barrier, the cornea, the male or female reproductive organs, the immune system, or any other organ of the body.

In some embodiments the cultured hepatocytes are in the form of a cell coculture that comprises hepatocytes and cells of at least one other type of cell. In some embodiments the cultured hepatocytes are in the form of a cell coculture that comprises at least hepatocytes and cells of a type that exhibit stromal characteristics in the coculture (stromal cells).

In some embodiments a hepatocyte-stromal cell coculture comprises hepatocytes and at least one stromal cell type disposed on the surface of a solid substrate. In some embodiments the hepatocyte-stromal cell culture is disposed on the surface of a solid substrate in a deliberately micropatterned configuration. In some embodiments the hepatocyte-stromal cell culture is disposed on the surface of a solid substrate in a configuration wherein the cells of the two types are interspersed or intermixed with each other. In some embodiments a hepatocyte-stromal cell coculture comprises hepatocytes and at least one stromal cell type that have together assumed a spheroidal configuration. In some embodiments a hepatocyte-stromal cell coculture comprises hepatocytes and at least one stromal cell type disposed within a solid lattice that enables the cells to assume and/or maintain their coculture configuration. In some embodiments of the hepatocyte-stromal cell coculture the hepatocytes and a single stromal cell type collectively represent at least about 95%, at least about 96%, at least about 97%, at least about 98%, at least about 99%, at least about 99.5%, at least about 99.9%, or at least about 99.99% of the cells in the coculture. In some embodiments of the hepatocyte-stromal cell coculture the hepatocytes and a single stromal cell type collectively represent less than any of the foregoing percentages of the total number of cells in the coculture.

Typically the hepatocytes and stromal cells are present in the coculture at a ratio of from 1:10 to 10:1. In some embodiments the hepatocytes and stromal cells are present in the coculture at a ratio of from 2:10 to 10:2. In some embodiments the hepatocytes and stromal cells are present in the coculture at a ratio of from 2:10 to 4:10. In some embodiments the hepatocytes and stromal cells are present in the coculture at a ratio of from 4:10 to 6:10. In some embodiments the hepatocytes and stromal cells are present in the coculture at a ratio of from 6:10 to 8:10. In some embodiments the hepatocytes and stromal cells are present in the coculture at a ratio of from 8:10 to 1:1. In some embodiments the hepatocytes and stromal cells are present in the coculture at a ratio of from 1:1 to 10:8. In some embodiments the hepatocytes and stromal cells are present in the coculture at a ratio of from 10:8 to 10:6. In some embodiments the hepatocytes and stromal cells are present in the coculture at a ratio of from 10:6 to 10:4. In some embodiments the hepatocytes and stromal cells are present in the coculture at a ratio of from 10:4 to 10:2. In some embodiments the hepatocytes and stromal cells are present in the coculture at a ratio of about 10:1, 10:2, 10:3, 10:4, 10:5, :10:6, 10:7, 10:8, 10:9, 1:1, 9:10, 8:10, 7:10, 6:10, 5:10, 4:10, 3:10, 2:10, or 1:10.

In some embodiments the hepatocyte-stromal cell coculture comprises at least two stromal cell types. In some embodiments the hepatocyte-stromal cell coculture comprises two stromal cell types that each represent at least about 0.01%, at least about 0.1%, at least about 0.5%, at least about 1%, at least about 2%, at least about 3%, at least about 4%, at least about 5%, at least about 10%, at least about 20%, at least about 30%, or at least about 40% of the cells in the coculture. In some embodiments the hepatocyte-stromal cell coculture comprises two stromal cell types that each represent materially different percentages of the total number of cells in the coculture. Some embodiments comprise more than two different stromal cell types. In some embodiments the functional contribution of all the stromal cell types in the coculture is principally stromal in nature. In some embodiments at least one stromal cell type provides a greater stromal contribution to the cell culture than does the at least a second stromal cell type. In some embodiments the at least one second stromal cell type makes a contribution to the over-all function or competency of the cell coculture that is not stromal in nature.

The hepatocytes in the hepatocyte-stromal cell coculture be any type of hepatocyte, including without limitation primary hepatocytes, hepatocyte cell lines, and hepatocytes formed by differentiating stem cells such as embryonic stem cells, adult stem cells, and/or induced pluripotent stem cells. In some embodiments all the hepatocytes in a hepatocyte-stromal cell coculture are primary hepatocytes. In some embodiments all the hepatocytes in a hepatocyte-stromal cell coculture are from at least one hepatocyte cell line. In some embodiments all the hepatocytes in a hepatocyte-stromal cell coculture are hepatocytes formed by differentiating stem cells such as embryonic stem cells, adult stem cells, and/or induced pluripotent stem cells. In some embodiments the hepatocytes in a hepatocyte-stromal cell coculture are a mixture of hepatocytes comprising primary hepatocytes and at least one hepatocyte cell line. In some embodiments the hepatocytes in a hepatocyte-stromal cell coculture are a mixture of hepatocytes comprising at least one hepatocyte cell line and hepatocytes formed by differentiating stem cells such as embryonic stem cells, adult stem cells, and/or induced pluripotent stem cells. In some embodiments the hepatocytes in a hepatocyte-stromal cell coculture are a mixture of hepatocytes comprising primary hepatocytes and hepatocytes formed by differentiating stem cells such as embryonic stem cells, adult stem cells, and/or induced pluripotent stem cells. In some embodiments the hepatocytes in a hepatocyte-stromal cell coculture are a mixture of hepatocytes comprising primary hepatocytes, at least one hepatocyte cell line, and hepatocytes formed by differentiating stem cells such as embryonic stem cells, adult stem cells, and/or induced pluripotent stem cells.

In some embodiments the hepatocyte-stromal cell coculture comprises hepatocytes from a single donor mammal. Thus, in some embodiments the hepatocyte-stromal cell coculture comprises primary hepatocytes all obtained from a single donor mammal. In some embodiments the hepatocyte-stromal cell coculture comprises hepatocytes from a plurality of hepatocyte cell lines, wherein the plurality of cell lines are all obtained from cells of a single donor mammal. In some embodiments the hepatocytes in a hepatocyte-stromal cell coculture are hepatocytes formed by differentiating stem cells such as embryonic stem cells, adult stem cells, and/or induced pluripotent stem cells that all anise from a single donor mammal.

In some embodiments the hepatocyte-stromal cell coculture comprises hepatocytes from a plurality of donor mammals. Thus, in some embodiments the hepatocyte-stromal cell coculture comprises primary hepatocytes obtained from a plurality of donor mammals. In some embodiments the primary hepatocytes are a mixture of lots obtained from 2, 3, 4, 5, 6, 7, 8, 9, or 10 different donor mammals of the same species. In some embodiments the primary hepatocytes are a mixture of lots obtained from 2, 3, 4, 5, 6, 7, 8, 9, or 10 different donor mammals of more than one species. In some embodiments the hepatocyte-stromal cell coculture comprises hepatocytes from a plurality of hepatocyte cell lines, wherein the plurality of cell lines are obtained from cells of a plurality of donor mammals. In some embodiments the hepatocyte cell lines comprise cell lines obtained from 2, 3, 4, 5, 6, 7, 8, 9, or 10 different donor mammals. In some embodiments the hepatocytes in a hepatocyte-stromal cell coculture are hepatocytes formed by differentiating stem cells such as embryonic stem cells, adult stem cells, and/or induced pluripotent stem cells that all anise from a plurality of donor mammal. In some embodiments the hepatocyte cell lines comprise hepatocytes formed by differentiating stem cells from 2, 3, 4, 5, 6, 7, 8, 9, or 10 different donor mammals.

The hepatocytes may be those of any mammal. In some embodiments the hepatocytes are of a mammal selected from a human, a non-human primate (such as a cynomolgus monkey), a farm animal (such as pig, horse, cow, and sheep), a domestic mammal (such as dogs, cats, guinea pig, mini-pig, and rabbit), and rodents (such as mice and rats).

In a preferred embodiment the hepatocytes are primary hepatocytes. Primary hepatocytes may (but need not be) supplied in cryopreserved form. Cryopreserved human hepatocytes may be obtained from commercial enterprises such as Thermo Fisher (formerly, Life Technologies Corporation) and BioreclamationlVT, among others. Cryopreserved non-human primate hepatocytes may be obtained from commercial enterprises such as Thermo Fisher (formerly, Life Technologies Corporation) and BioreclamationlVT, among others. Cryopreserved dog hepatocytes may be obtained from BioreclamationlVT. Cryopreserved rat hepatocytes may be obtained from commercial enterprises such as Thermo Fisher (formerly, Life Technologies Corporation) and BioreclamationlVT, among others. In some embodiments the stromal cell type is from the same type of mammal as the hepatocyte. In some embodiments the stromal cell type is from a different type of mammal than the hepatocyte.

In some embodiments the hepatocyte-stromal cell coculture comprises a third cell type. In some embodiments the third cell type is a stromal cell. In some embodiments the third cell type is not a stromal cell. In some embodiments the third cell type is a parenchymal cell. In some embodiments the third cell type is not a non-parenchymal cell. In some embodiments the third cell type is selected from Ito cells, endothelial cells, biliary duct cells, immune-mediating cells, and stem cells. In some embodiments, the immune-mediating cells are selected from macrophages, T cells, neutrophils, dendritic cells, mast cells, eosinophils and basophils.

In some embodiments the third cell type is a Kuppfer cell. In some embodiments the Kuppfer cells represent at least about 0.01%, at least about 0.1%, at least about 0.5%, at least about 1%, at least about 2%, at least about 3%, at least about 4%, at least about 5%, at least about 10%, or more than at least 10% of the cells in the coculture.

In some embodiments the stromal cell type is an endothelial cell. In some embodiments the stromal cell type is a fibroblast cell. In some embodiments the stromal cell is a primary cell. In some embodiments the stromal cell is obtained from a cell line. In some embodiments the stromal cell is a transformed cell. In some embodiments the stromal cell is differentiated in vitro from a stem cell, such as an embryonic stem cell, adult stem cell, or induced pluripotent stem cell. Numerous sources of stromal cells such as fibroblasts are known in the art and may be utilized in the hepatocyte-stromal cell cocultures. One example is the NIH 3T3-J2 cell line. (See for example US 2013/0266939 A1.)

The art teaches that some aspects of hepatocyte function in culture are improved by disposing hepatocytes and stromal cells onto a solid substrate such that the hepatocytes are attached to the substrate in a first step in a cellular island configuration. (See US 2013/0266939 A1.) Specifically, such methods rely on formation of cellular islands of hepatocytes on a substrate, the hepatocyte islands surrounded by a non-parenchymal cell type such as a stromal cell type. The hepatocyte islands are formed by first placing an extracellular matrix component or derivative onto a solid substrate in an island pattern and then allowing the hepatocytes to adhere to the extracellular matrix component or derivative. The non-parenchymal cell type is then added and allowed to “fill in” the portions of the substrate that don't contain hepatocytes. A fundamental feature of such systems is that the hepatocytes are not dispersed across the substrate surface.

In some embodiments of the hepatocyte-stromal cell coculture of this invention hepatocytes are distributed in a cellular island configuration such as described in US 2013/0266939 A1. However, in preferred embodiments the hepatocytes are substantially dispersed and intermixed with stromal cells across the surface of the solid substrate. In some preferred embodiments the hepatocytes are substantially dispersed and intermixed with stromal across the surface of the solid substrate, and the hepatocytes and stromal cells have reorganized themselves such that their configuration comprises at least some of the hepatocytes being configured partially or completely on top of at least some of the stromal cells, and/or some of the stromal cells being configured partially or completely on top of at least some of the hepatocytes.

As used herein, “dispersed across the surface” in reference to an arrangement of hepatocytes on a solid support in a hepatocyte-stromal cell coculture means that at least one of the following criteria applies to the coculture: 1) at least about 20%, at least about 30%, at least about 40%, or at least about 50% of the surface of the solid substrate is covered by at least one hepatocyte; 2) at least about 2%, at least about 5% or at least about 10% of the hepatocytes in the coculture are located on top of a stromal cell that is in contact with the solid substrate; and 3) the hepatocytes were not seeded onto the solid substrate by adding the hepatocytes to a solid substrate comprising islands of at least one extracellular matrix component to create islands of hepatocytes attached to the solid substrate. Note that a single hepatocyte may be counted as meeting both criterion 1 and criterion 2.

In preferred embodiments the metabolic function of the hepatocyte-stromal cell coculture is long-enduring. In some embodiments the time period of endurance of the culture is for at least one day, at least two days, at least three days, at least five days, at least seven days, at least ten days, at least fourteen days, at least twenty-one days, at least twenty-eight days, at least thirty-five days, at least forty-two days, or more than at least forty-two days. In some embodiments the function of the hepatocyte-stromal cell coculture is determined by measuring an activity selected from gene expression, cell function, metabolic activity, morphology, and/or a combination thereof, of the hepatocytes in the coculture. In some embodiments the function of the hepatocyte-stromal cell coculture is determined by measuring the level of expression and/or activity of at least one CYP450 enzyme. The level of metabolic expression and/or metabolic activity of at least one CYP450 enzyme may be measured by measuring expression of the CYP450 enzyme mRNA, by measuring expression of the CYP450 enzyme protein, or by a functional assay of CYP450 enzyme activity. In some embodiments, the metabolic activity is a so-called Phase I metabolic enzyme activity such as a CYP450 enzyme activity. In some embodiments, the CYP450 enzyme is a CYP450 enzyme selected from CYP1A2, CYP1B1, CYP2A6, CYP2B6, CYP2C, CYP2D6, CYP2E1, CYP2F1, CYP2J2, CYP3A4, CYP4A, and CYP4B. In some embodiments, the Phase I metabolic enzyme activity is a non-CYP enzyme activity, such as MAO activity. In some embodiments, the metabolic activity is a so-called Phase II metabolic enzyme activity such as a UGT or SULT enzyme activity.

The metabolic function of the hepatocyte-stromal cell coculture is considered long enduring if the metabolic function of the coculture endures longer in the hepatocyte-stromal cell coculture than the metabolic function of a control hepatocyte monoculture. In some embodiments the metabolic function of the coculture endures for at least three days. In some embodiments the metabolic function of the coculture endures for at least four days. In some embodiments the metabolic function of the coculture endures for at least seven days. In some embodiments the metabolic function of the coculture endures for at least fourteen days. In some embodiments the metabolic function of the coculture endures for at least twenty-one days. In some embodiments the metabolic function of the coculture endures for at least twenty-eight days. In some embodiments the metabolic function of the coculture endures for at least thirty-five days. In some embodiments the metabolic function of the coculture endures for at least forty-two days. In some embodiments the metabolic function of the coculture endures for longer than at least forty-two days.

In some embodiments the coculture is cultured in serum-free or essentially serum-free media. In some embodiments the coculture is cultured in media containing serum. In some embodiments the media comprises about 0.1% serum, about 0.2% serum, about 0.3% serum, about 0.4% serum, about 0.5% serum, about 0.6% serum, about 0.7% serum, about 0.8% serum, about 0.9% serum, about 1% serum, about 2% serum, about 3% serum, about 4% serum, about 5% serum, about 6% serum, about 7% serum, about 8% serum, about 9% serum, or about 10% serum. In some embodiments the media comprises at least about 0.1% serum, at least about 0.2% serum, at least about 0.3% serum, at least about 0.4% serum, at least about 0.5% serum, at least about 0.6% serum, at least about 0.7% serum, at least about 0.8% serum, at least about 0.9% serum, at least about 1% serum, at least about 2% serum, at least about 3% serum, at least about 4% serum, at least about 5% serum, at least about 6% serum, at least about 7% serum, at least about 8% serum, at least about 9% serum, or at least about 10% serum. In some embodiments the media comprises less than or equal to about 0.1% serum, less than or equal to about 0.2% serum, less than or equal to about 0.3% serum, less than or equal to about 0.4% serum, less than or equal to about 0.5% serum, less than or equal to about 0.6% serum, less than or equal to about 0.7% serum, less than or equal to about 0.8% serum, less than or equal to about 0.9% serum, less than or equal to about 1% serum, less than or equal to about 2% serum, less than or equal to about 3% serum, less than or equal to about 4% serum, less than or equal to about 5% serum, less than or equal to about 6% serum, less than or equal to about 7% serum, less than or equal to about 8% serum, less than or equal to about 9% serum, or less than or equal to about 10% serum.

In some embodiments of this disclosure the systems and methods provide comparable hepatocyte-stromal cell cocultures from at least three of four different mammalian species.

The embodiments presented in the examples utilize certain primary hepatocyte-stromal cell cocultures. However, the invention should not be understood as limited to that configuration of the cell culture. The full scope of the invention encompasses any in vitro cell culture system that maintains cells (e.g., hepatocytes) in a state of high metabolic functionality over an extended culture period such that test compounds may be incubated with the cell culture over first and second culture periods so that toxicity over the first and second culture periods may be compared.

In some embodiments a different cell type is substituted for the hepatocytes. For example, this invention includes without limitation embodiments that measure toxicities in cells from organs other than the liver (such as heart and kidney, as two examples).

### B. Methods of Characterizing the Time-Based Hepatotoxicity of a Test Compound

In some embodiments the invention provides methods of characterizing the time-based hepatotoxicity of a test compound. The test compound may be without limitation any type of compound, including a compound that has or is under investigation for a therapeutic purpose as well as environmental and industrial chemicals.

In some embodiments the methods comprise incubating a first in vitro culture comprising hepatocytes with a test compound for a first culture period. In some embodiments a plurality of first in vitro cultures comprising hepatocytes are incubated with a test compound for a first culture period. The test compound may be added to the first in vitro culture(s) (i.e., the first in vitro culture(s) may be dosed with the test compound) at one or a plurality of timepoints during the first culture period. Typically, at least one feature of the culture differs between the plurality of first in vitro cultures. For example, different first in vitro cultures may be incubated in the presence of different concentrations of the test compound and/or by changing the frequency at which the test compound is dosed.

In some embodiments the methods comprise measuring at least one cytotoxic effect of the test compound on the hepatocytes of the first in vitro culture over the first culture period (such as, for example, measuring at the end of the first culture period) to thereby define the hepatotoxicity of the test compound over the first culture period. If the method utilizes a plurality of first in vitro cultures comprising hepatocytes incubated with a test compound for a first culture period at different concentrations of test compound then the method will typically comprise measuring the at least one cytotoxic effect of the test compound on the hepatocytes of each of the plurality of first in vitro cultures over the first culture period. In some embodiments a TC50 value is then determined for the test compound over the first culture period.

In some embodiments the methods further comprise incubating a second in vitro culture comprising hepatocytes with the test compound for a second culture period. The second culture period is typically of longer duration than the first culture period. In some embodiments the second culture period comprises a larger number of individual dosings of test compound than does the first culture period. In some embodiments a plurality of second in vitro cultures comprising hepatocytes are incubated with the test compound for a second culture period. The test compound may be added to the second in vitro culture(s) at one or a plurality of timepoints during the second culture period. Typically, at least one feature of the culture differs between the plurality of second in vitro cultures. For example, different second in vitro cultures may be incubated in the presence of different concentrations of the test compound and/or by changing the frequency at which the test compound is dosed.

In some embodiments the methods comprise measuring at least one cytotoxic effect of the test compound on the hepatocytes of the second in vitro culture over the second culture period (such as, for example, at the end of the second culture period) to thereby define the hepatotoxicity of the test compound over the second culture period. If the method utilizes a plurality of second in vitro cultures comprising hepatocytes incubated with a test compound for a second culture period at different concentrations of test compound then the method will typically comprise measuring the at least one cytotoxic effect of the test compound on the hepatocytes of each of the plurality of second in vitro cultures over the second culture period. In some embodiments a TC50 value is then determined for the test compound over the second culture period. In some embodiments the methods comprise measuring at least one cytotoxic effect of the test compound on hepatocytes of at least one additional in vitro culture at the end of at least one additional culture period (i.e., a culture period of duration even longer than the duration of the second culture period) to thereby define the hepatotoxicity of the test compound over the third culture period

Typically the hepatotoxicity of the test compound over the first and second culture periods is defined as the TC50 value for the test compound, determined at completion of the first and second culture periods. In some embodiments the number of cells in the culture that are alive and/or dead is determined by a method of counting live and/or dead cells. In some embodiments a method of measuring the extent and/or rate of occurrence of at least one toxicity process is used. In some embodiments the hepatotoxicity of the test compound measured at the end of each such culture period is defined as the ratio of the maximum concentration that the test compound reaches in the plasma of a human or other species of mammal to which a particular dosage is administered (known as the “C.” concentration) divided by the TC₅₀ value, signified as TC₅₀/Cₘₐₓ.

The hepatotoxicity of the test compound over the first culture period is compared to the hepatotoxicity of the test compound over the second culture period to thereby characterize the time-based hepatotoxicity of the test compound. In some embodiments this comparison is a simple arithmetic ratio of a degree of toxicity of the test compound over the first culture period to the degree of toxicity of the test compound over the second culture period. In some embodiments the hepatotoxicity of the test compound over the second culture period is greater than the hepatotoxicity of the test compound over the first culture period and the test compound is identified as exhibiting time-based hepatotoxicity such that the arithmetic computation of the TC50 value measured at the end of the first (i.e, shorter-enduring) culture period divided by the TC50 value measured at the end of the second (i.e, longer-enduring) culture period is a number greater than one (1). This invention is based in part on the surprising observation of the inventors that this comparison is a more accurate predictor of in vivo toxicity of test compounds than a simple measurement of toxicity measured at the end of the second (i.e., longer-enduring) culture period. This result was unexpected and is contrary to conventional wisdom in this field.

In some embodiments the computed ratio, difference or variation between the hepatotoxicity of the test compound measured at the end of the second culture period relative to the hepatotoxicity of the test compound measured at end of the first culture period is compared to a defined threshold in order to determine whether a defined risk of in vivo toxicity is observed. In some embodiments the threshold is a predefined threshold. In some embodiments the threshold is an absolute number (for example a ratio value of at least 4) while in other embodiments the threshold is a multiple of a ratio determined experimentally for a control compound.

In some embodiments the hepatotoxicity of the test compound over the second culture period is greater than the hepatotoxicity of the test compound over the first culture period by at least a pre-defined threshold and the test compound is identified as exhibiting time-based hepatotoxicity. In some embodiments the hepatotoxicity of the test compound over the second culture period is not greater than the hepatotoxicity of the test compound over the first culture period by at least a pre-defined threshold and the test compound is identified as not exhibiting time-based hepatotoxicity.

In some embodiments the hepatotoxicity of the test compound over the second culture period is greater than the hepatotoxicity of the test compound over the first culture period by a factor of at least 2, at least 3, at least 4, at least 5, at least 6, at least 7, at least 8, at least 9 or at least 10. This factor may be an arithmetically computed ratio, a subtracted difference between logarithmically derived numbers, or any other form of computed measurement.

In some embodiments the first culture period is from twelve hours to five days. In some embodiments the first culture period is from twelve to thirty-six hours. In some embodiments the first culture period is from one day to five days, from one day to three days, or from three days to five days. In some embodiments the first culture period is from two days to four days. In some embodiments the first culture period is one, two, three, four, or five days. In some embodiments the first culture period is less than five days. In some embodiments the first culture period is from ten to sixty minutes. In some embodiments the first culture period is from sixty minutes to twelve hours. In a preferred embodiment the first culture period is one day or from twelve to thirty six hours.

In some embodiments the second culture period is from three days to twenty-eight days. In some embodiments the second culture period is from seven to twenty-eight days. In some embodiments the second culture period is longer than twenty-eight days. In some embodiments the third culture period is longer than the second culture period. In some embodiments the second culture period is from seven to fourteen days. In some embodiments the second culture period is from fourteen to twenty-one days. In some embodiments the second culture period is from twenty-one days to twenty-eight days. In some embodiments the second culture period is from fourteen days to twenty-eight days. In some embodiments the second culture period is one, two, three, four, five, six, seven, eight, nine, ten, eleven, twelve, thirteen, or fourteen days, and is longer than the first culture period. In some embodiments the second culture period is from ten to sixty minutes, and is longer than the first culture period. In some embodiments the second culture period is from sixty minutes to twelve hours, and is longer than the first culture period.

The second culture period is longer than the first culture period. In some embodiments the second culture period is longer than the first culture period by three, five, seven, nine, eleven, or fourteen days, or by more than fourteen days.

The third culture period is longer than the second culture period. In some embodiments the third culture period is longer than the second culture period by one, two, three, five, seven, nine, eleven, or fourteen days, or by more than fourteen days.

This invention includes embodiments that utilize simple arithmetic, multivariate algebraic, integral, differential, probabilistic, stochastic, and simulative forms of measurement and/or computational models.

### C. Methods of Measuring a Cytotoxic Effect of a Test Compound

In some embodiments the cytotoxic effect of the test compound is cell death.

In some embodiments the cytotoxic effect of the test compound is a cytotoxic process leading to cell death.

In some embodiments the methods comprise use of a plurality of first cultures and different concentrations of test compound in order to derive an LC50.

In some embodiments the cytotoxic effect is a cell attribute associated with cytotoxicity. In some embodiments the cell attributes are selected from cell adhesion to a substrate, intracellular enzymatic conversion of a redox dye into a detectable end product, mitochondrial membrane potential formation of reactive oxygen species (ROS), release of intracellular enzyme into media, protein secretion, cellular byproduct secretion, and oxygen consumption rate by hepatocytes in culture.

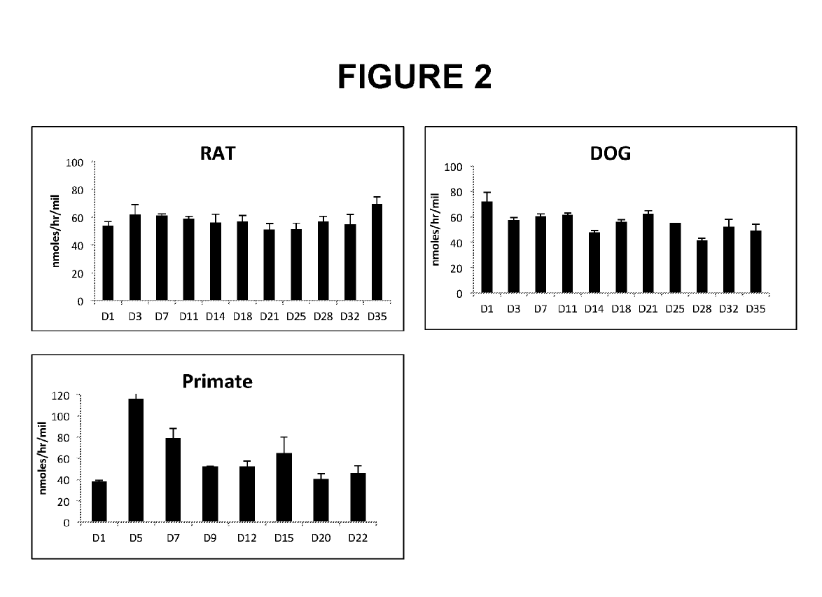

Cell adhesion to a substrate may be assessed using any method known in the art. In some embodiments cell adhesion is assessed using the xCELLigence RTCA SP System manufactured by ACEA Biosciences, Inc. in conjunction with the a hepatocyte cell culture provided herein. The hepatocyte culture is established in a suitable culture substrate such as in an E-Plate View 96 plates manufactured by ACEA Biosciences, Inc. The presence of the cells on top of the electrodes in the E-plate will affect the local ionic environment at the electrode/solution interface, leading to an increase in the impedance of the electrical system comprising the electrode, the solution and the cells. The more cells are attached on the electrodes, the larger the increases in electrode impedance. In addition, the impedance depends on the quality of the cell interaction with the electrodes. For example, increased cell adhesion or spreading will lead to a larger change in electrode impedance. Thus, electrode impedance, which is displayed as cell index (CI) values, can be used to monitor cell viability, number, morphology, and adhesion in the coculture. Intracellular enzymatic conversion of a redox dye into a detectable end product may be assessed using any method known in the art. In some embodiments the CellTiter-Blue® Cell Viability Assay from Promega is used. The CellTiter-Blue®Cell Viability Assay provides a homogeneous, fluorometric method for estimating the number of viable cells present in multiwell plates. The simple protocol involves adding a single reagent directly to cells cultured in serum-supplemented medium. The CellTiter-Blue® Assay is based on the ability of living cells to convert a redox dye (resazurin) into a fluorescent end product (resorufin). Viable cells retain the ability to reduce resazurin into resorufin. Nonviable cells rapidly lose metabolic capacity, do not reduce the indicator dye, and thus do not generate a fluorescent signal. The CellTiter-Blue® Reagent is a buffered solution containing highly purified resazurin. The ingredients have been optimized for use as a cell viability assay. The spectral properties of CellTiter-Blue® Reagent change upon reduction of resazurin to resorufin (FIG. 2). Resazurin is dark blue in color and has little intrinsic fluorescence until it is reduced to resorufin, which is pink and highly fluorescent (579Ex/584Em). The visible light absorbance properties of CellTiter-Blue® Reagent undergo a “blue shift” upon reduction of resazurin to resorufin. The absorbance maximum of resazurin is 605 nm and that of resorufin is 573 nm. Either fluorescence or absorbance may be used to record results; however, fluorescence is the preferred method because it is more sensitive and involves fewer data calculations.

Formation of reactive oxygen species (ROS) may be assessed using any method known in the art. In some embodiments the ROS-Glo™ H₂O₂ Assay from Promega is used. The ROS-Glo™ H₂O₂ Assay is a homogeneous, fast and sensitive bioluminescent assay that measures the level of hydrogen peroxide (H₂O₂), a reactive oxygen species (ROS), directly in cell culture or in defined enzyme reactions. A derivatized luciferin substrate is incubated with sample and reacts directly with H₂O₂ to generate a luciferin precursor. Addition of ROS-Glo™ Detection Solution converts the precursor to luciferin and provides Ultra-Glo™ Recombinant Luciferase to produce light signal that is proportional to the level of H₂O₂ present in the sample. The assay can be performed in various cell culture media with or without serum, eliminating the need to remove the media from cultured cells before performing the assay. The homogeneous assay is performed following a simple two-reagent-addition protocol that does not require sample manipulation. The assay can be completed in less than 2 hours after reagent addition. The ROS-Glo™ H₂O₂ Substrate reacts directly with H₂O₂, obviating the need for horseradish peroxidase (HRP) as a coupling enzyme and thus eliminating false hits associated with HRP inhibition.

Assays that detect release of an intracellular enzyme into media may be based on any suitable enzyme and utilize suitable reagents known in the art. In some embodiments the enzyme is adenylate kinase (AK). The ToxiLight™ BioAssay Kit from LONZA is one suitable example. It is a bioluminescent, non-destructive cytolysis assay kit designed to measure the release of the enzyme, adenylate kinase (AK), from damaged cells. AK is a robust protein present in all eukaryotic cells, which is released into the culture medium when cells die. The enzyme actively phosphorylates ADP to form ATP and the resultant ATP is then measured using the bioluminescent firefly luciferase reaction. As the level of cytolysis increases, the amount of AK in the supernatant also increases, which results in emission of higher light intensity by the ToxiLight™ reagent. Because the ToxiLight™ BioAssay Kit exploits the fact that AK is released from cells when they die, there is no need for cell lysis (unlike many other cytotoxicity assays). Repeated samples of supernatant can therefore be taken over time without disrupting the cells themselves. This allows for kinetic analysis of cell death. The ToxiLight™ BioAssay Kit also facilitates high content screening by allowing other tests to be performed on the original cells. All the components required for AK detection are contained within a single reagent making the assay very simple to perform. The kit provides outstanding sensitivity with a detection limit of 10 cells per microwell with a dynamic range of over 5 orders of magnitude. Extended signal stability also makes the assay suitable for batch processing in high-throughput screening applications. The ToxiLight™ BioAssay Kit exploits the cyclic nature of the AK reaction whereby a small amount of the AK enzyme can generate high concentrations of ATP. This enables the detection of very subtle changes in cytotoxicity and reduces false negatives. ToxiLight™ assay detects cellular AK present in cell culture supernatants, eliminating the need to lyse cells to perform the assay, and allowing multiple tests to be performed on the same sample. The assay requires the simple addition of a single reagent, which can be added directly to wells in which cells are growing, or to a small sample of aspirated culture supernatant.

The oxygen consumption rate by hepatocytes in culture may be assessed using any suitable assay known in the art. One examples is the XF technology and stress test kits from Seahorse Biosciences. The kits measure energy utilization in living cells, simultaneously quantifying mitochondrial respiration and glycolysis in a microplate, in real-time. XF technology offers a robust and simple method for studying substrate utilization, mitochondrial function, energy expenditure and cell quality in micro plates, without the use of large number of cells, flasks, electrodes, dyes, radioactive materials or lysis of cells that is typical of other methods. The XF Analyzer's unique ability to measure the metabolic phenotype of cells by simultaneously measuring respiration and glycolysis in real-time, and the shift between the two pathways under pathological states, enables one to connect physiological traits of cells with toxicity induced by a molecular entity. The XF^{e} Analyzer measures oxygen consumption rate (OCR) and extracellular acidification rate (ECAR) at intervals of approximately 2-5 minutes. OCR is an indicator of mitochondrial respiration, and ECAR is predominantly the result of glycolysis. Real-time measurements of OCR and ECAR are made by isolating an extremely small volume (less than 7 μl) of medium above a monolayer of cells within a microplate. Cellular oxygen consumption (respiration) and proton excretion (glycolysis) causes rapid, easily measurable changes to the concentrations of dissolved oxygen and free protons in this “transient microchamber” which are measured every few seconds by solid state sensor probes residing 200 microns above the cell monolayer. The instrument continues to measure the concentrations until the rate of change is linear and then calculates the slope to determine OCR and ECAR, respectively. Once a measurement is completed, the probes lift which allows the larger medium above to mix with the medium in the transient microchamber, restoring cell values to baseline. An integrated drug delivery system allows sequential addition of up to four drugs per well at user-defined intervals. Prior to the start of the XF assay, cells are seeded onto a suitable substrate, such as the wells of an XF cell culture microplate. Seahorsesupplied, bicarbonate-free medium is typically used during the assay; however, it is contemplated that alternative media may also be used to coordinate the performance of multiple different assays per well. Up to two or four drugs may be added to the assay cartridge for use during the assay. Because XF measurements are non-destructive, the metabolic rate of the same cell population can be measured repeatedly over time while up to two or four different drugs can be injected sequentially into each well. Upon completion of an XF assay, other types of biological assays such as cell viability can be performed on the same plate. Total assay time is typically 35 to 90 minutes. To maintain normal cell physiology, a temperature control system maintains the XF Analyzer's internal environment at 37° C.

Another technology that may be used to measure oxygen consumption rate by hepatocytes in culture is the Mitoxpress Xtra offered commercially by Luxcel. MitoXpress®-Xtra (MitoXpress-Xtra & MitoXpress-Xtra-HS) assays measure extracellular oxygen consumption by cell populations.

In some embodiments the cell attributes characterized in the assay comprise or consist of two, three, four, five, six or seven of cell adhesion to a substrate, intracellular enzymatic conversion of a redox dye into a detectable end product, formation of reactive oxygen species (ROS), release of intracellular enzyme into media, protein secretion, excretion of cellular byproducts, and oxygen consumption rate by hepatocytes in culture. Each permutation of two, three, four, five, six and seven of these seven cell attributes is independently a specific embodiments of this invention.

In some embodiments the assay further comprises assaying albumen secretion and/or urea excretion.

In some embodiments the systems and methods comprise toxicity assays performed on at least three of six mammalian species. The six species may be selected from human, non-human primate, dog, and rat, mouse, and mini-pig.

### Examples

The following examples serve to more fully describe the manner of using the invention. These examples are presented for illustrative purposes and should not serve to limit the true scope of the invention.

### Example 1: Primary Hepatocyte-Stromal Cell Cocultures

Cryopreserved human hepatocytes were obtained from Thermo Fisher (formerly Life Technologies Corporation). Cryopreserved non-human primate (cynomolgus monkey) hepatocytes were obtained from Thermo Fisher (formerly Life Technologies Corporation). Cryopreserved dog hepatocytes were obtained from IVT Bioreclamation. Cryopreserved rat hepatocytes were obtained from Thermo Fisher (formerly Life Technologies Corporation). The cryopreserved hepatocytes were removed from liquid nitrogen and thawed. After thawing, cells were re-suspended in medium and cell number and cell viability was determined using trypan blue exclusion. Stromal cells were passed in a CO2 incubator until used for experimental plating. On plating day cells were detached from the plate, washed, and re-suspended in medium. Cell number and viability were determined using trypan blue exclusion.

Hepatocytes and stromal cells were seeded into collagen-coated 96-well plates at a density of 30,000 hepatocytes per well or in different sized wells at a comparable density. The hepatocytes were substantially dispersed across the surface of the well. The stromal cells were growth arrested prior to seeding.

The results of several experiments demonstrating the utility of the hepatocyte-stromal cell cocultures are presented in the figures attached hereto. (Figure numbers are provided in the lower right corner of the figures.)

### Example 2: Stable and Enduring Functionality of Primary Hepatocyte-Stromal Cell Cocultures

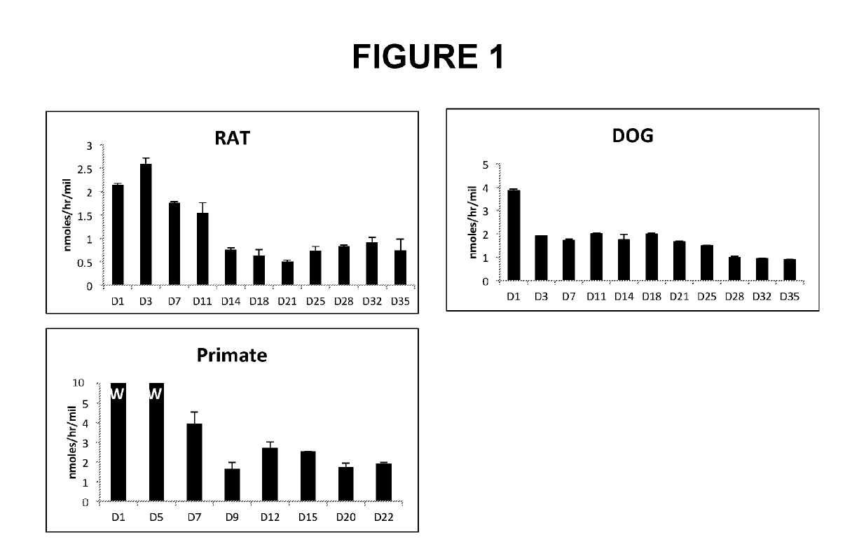

The data reported in FIGS. 1-5 shows the stable, high functionality of the hepatocyte-stromal cell cocultures described in Example 1. Human metabolic activity was characterized by CYP specific metabolite formation. CYP 3A4 activity was measured by the rate of midazolam conversion to 1-OH midazolam. CYP 2D6 activity was measured by the rate of dextromethorphan conversion to dextrophan. CYP 2C9 activity was measured by the rate of tolbutamide conversion to 4-OH tolbutamide. Dog, Rat and Monkey metabolic activity was characterized by formation of primary and secondary metabolites of coumarin. Primary metabolism was measured by the rate of formation of 7-hydroxycoumarin and secondary metabolism was measured by the formation of 7-hydroxycoumarin glucuronide and 7-hydroxycoumarinsufate. The concentrations of the metabolites were monitored over one hour in culture and analyzed via LC/MS/MS analysis. Overall, the results show that metabolic function can be maintained above acceptable levels for an extended period of time.

FIG. 1 shows that phase I enzyme function is long enduring in rat, dog, and primate hepatocyte-stromal cell cocultures of the invention. The rate of 7-hydroxycoumarin formation was measured at the indicated timepoints. Different patterns are evident for the different species; however, in each case phase-I enzyme function is long enduring. Rat and dog have phase I activity for at least 35 days and primate for at least 22 days from the day the hepatocytes are seeded.

FIG. 2 shows that phase II enzyme function is long enduring in rat, dog, and primate hepatocyte-stromal cell cocultures of the invention. The rate of 7-hydroxycoumaringlucuronide formation was measured at the indicated timepoints. Different patterns are evident for the different species; however, in each case phase-II enzyme function is long enduring. Rat and dog have phase II activity for at least 35 days and primate for at least 22 days from the day the hepatocytes are seeded.

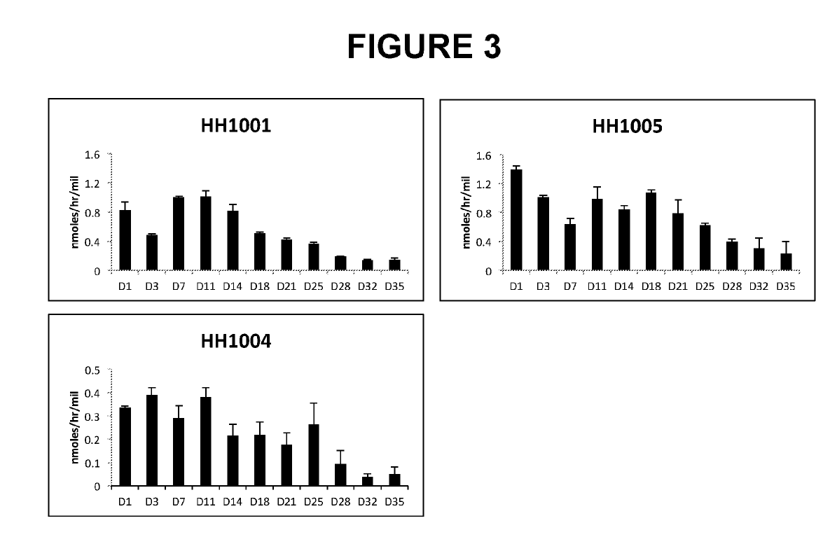

FIG. 3 shows long enduring CYP 3A4 function in hepatocyte-stromal cell cocultures made from three different lots of primary human hepatocytes. CYP 3A4 function was analyzed by measuring the rate of 1-hydroxymidazolam formation. Some lot to lot variation in baseline was observed, but all lots have 3A4 activity for 35 days from the day the cells are seeded.

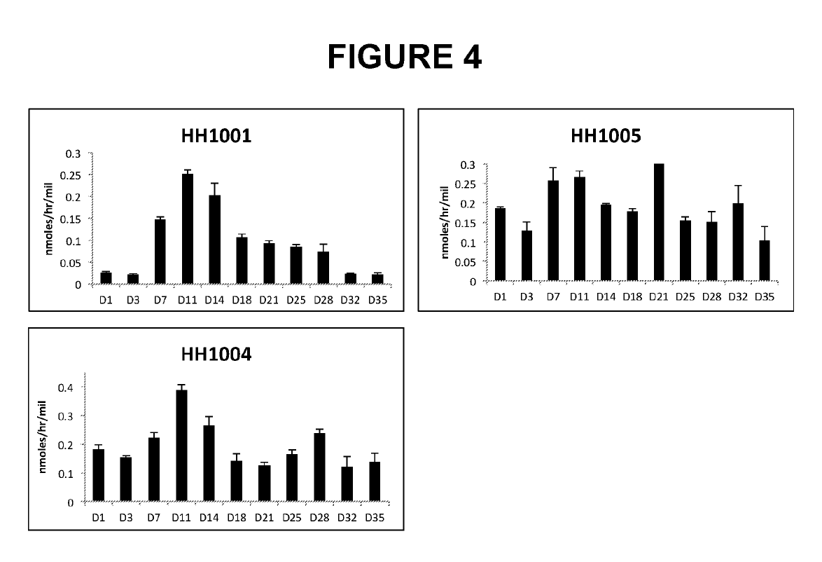

FIG. 4 shows long enduring CYP 2C9 function in hepatocyte-stromal cell cocultures made from three different lots of primary human hepatocytes. CYP 2C9 function was analyzed by measuring the rate of 4-hydroxytolbutamide formation. Some lot to lot variation in baseline was observed, but all lots have 2C9 activity for 35 days from the day the cells are seeded.

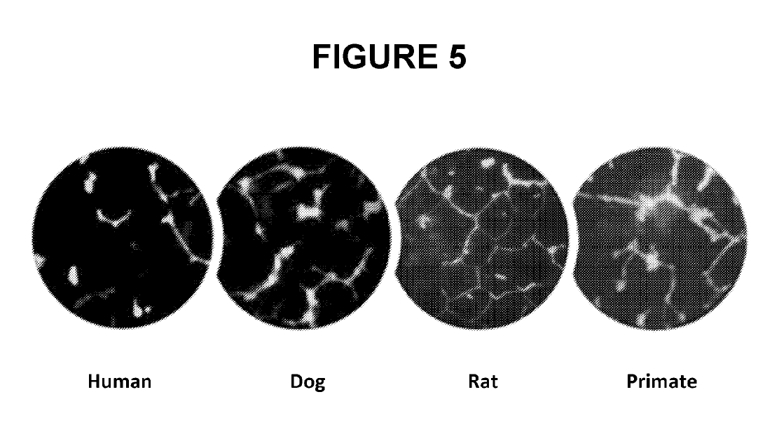

FIG. 5 shows staining of canaliculi in hepatocyte-stromal cell cocultures established using primary human, dog, rat, and primate hepatocytes.

### Example 3: Extended Window of High Functional Stability

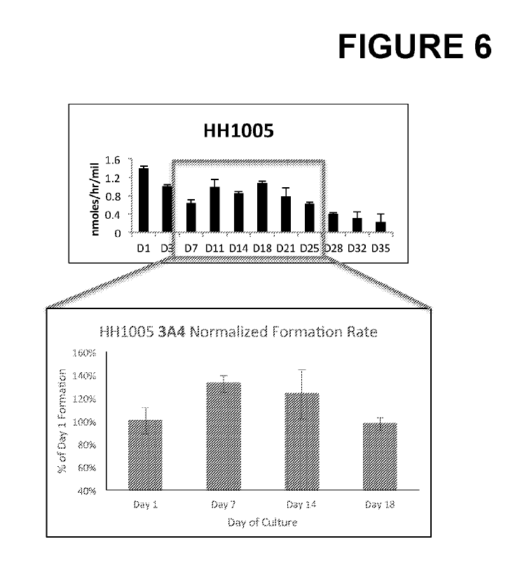

The data reported in FIGS. 6-9 shows that the stable, high functionality of the hepatocyte-stromal cell cocultures described in Example 1 is present over an extended window of time that allows performance of two week experiments in a frame of very elevated metabolic functional level and narrow enzymatic activity fluctuations. Human metabolic activity was characterized by CYP specific metabolite formation. CYP 3A4 activity was measured by the rate of midazolam conversion to 1-OH midazolam. CYP 2D6 activity was measured by the rate of dextromethorphan conversion to dextrophan. CYP 2C9 activity was measured by the rate of tolbutamide conversion to 4-OH tolbutamide. Dog, Rat and Monkey metabolic activity was characterized by formation of primary and secondary metabolites of coumarin. Primary metabolism was measured by the rate of formation of 7-hydroxycoumarin and secondary metabolism was measured by the formation of 7-hydroxycoumarin glucuronide and 7-hydroxycoumarinsufate. The concentrations of the metabolites were monitored over one hour in culture and normalized to day 1 formation rates and analyzed via LC/MS/MS analysis. Overall, the results show that metabolic function can be maintained in a window of stability for an extended period of time and this allows an experimenter to do long term experiments without a drop off in cellular function.

FIG. 6 shows that hepatocyte CYP 3A4 activity is stable in human hepatocyte-stromal cell cocultures from days 7 to 25 days in culture. By day 7 the cultured primary hepatocytes have finished remodeling and stabilized function. Function remains stable through day 25.

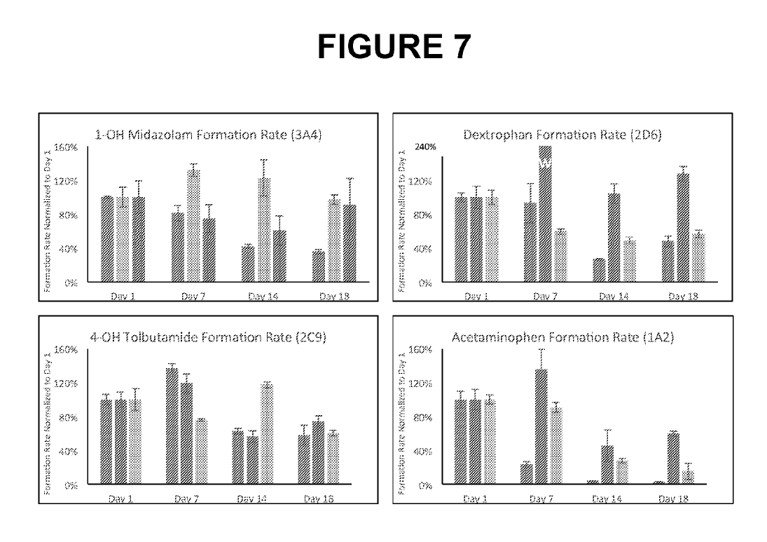

FIG. 7 shows that four different CYP enzymes remain stable between days 7 and 18 of culture in most or all of three different lots of human hepatocytes in human hepatocyte-stromal cell cocultures.

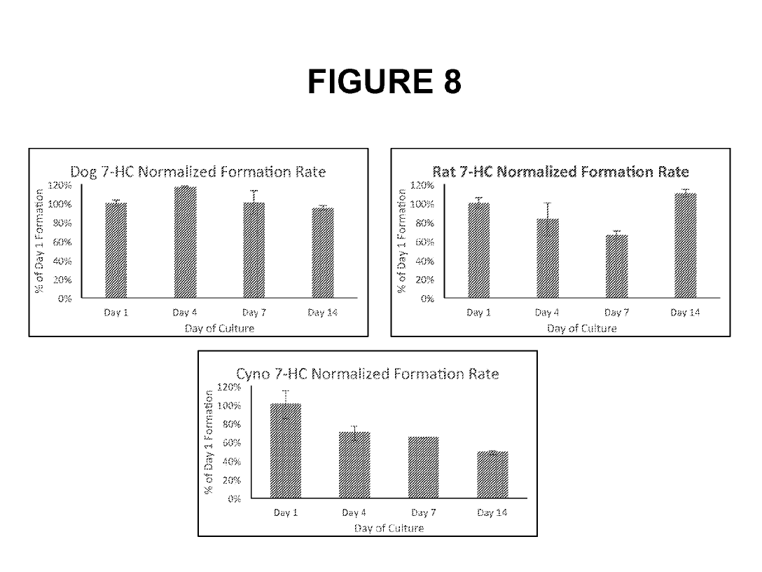

FIG. 8 shows a multi species window of stability for phase I metabolism.

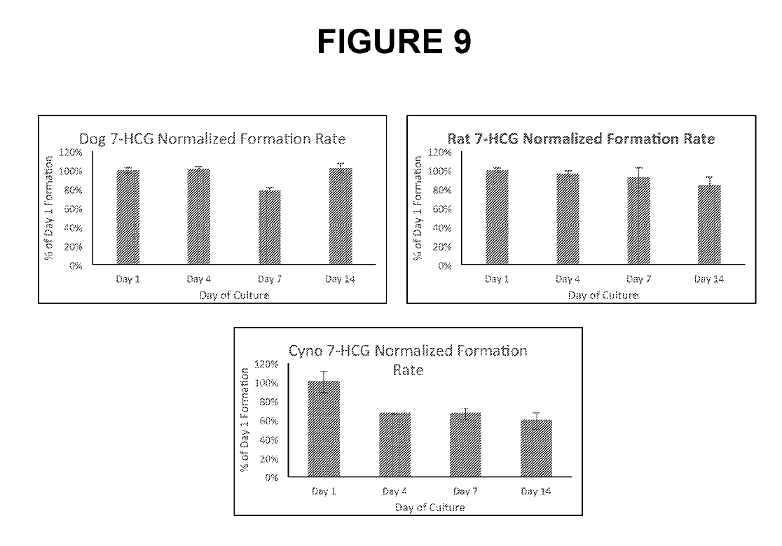

FIG. 9 shows a multi species window of stability for phase I metabolism.

### Example 4: Comparison of Primary Hepatocyte-Stromal Cell Cocultures to HepG2 Cells

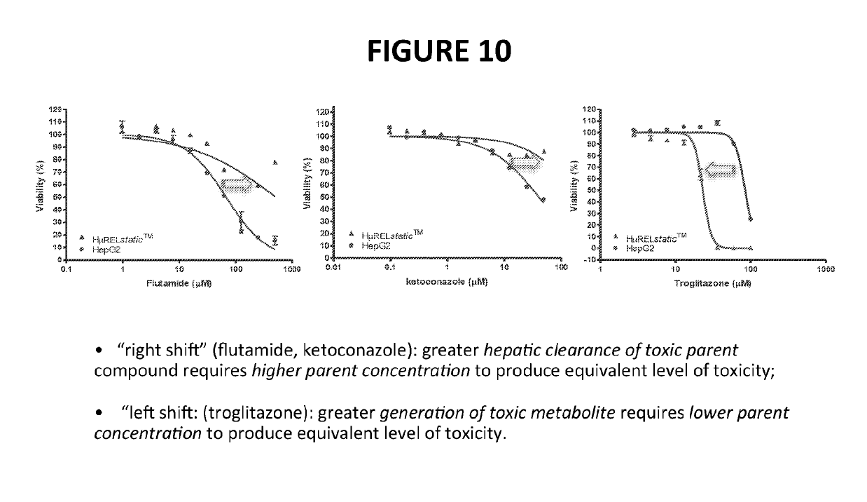

HepG2 cells were seeded into 96 well plates and allowed to become confluent for 24 hours. Primary human hepatocyte cocultures were established according to Example 1 and allowed to acclimate for 7 days before the experiment. At the start of the experiment, the cells were exposed to various concentrations of compound for four days with one repeat dose on the second day of the experiment. On the fourth day a the Promega ATP assay was run. The results are presented in FIG. 10. For flutamide and ketoconazole a right shift in the human hepatocyte coculture toxicity curve was observed as compared to the HepG2 cell toxicity curve. This appears to reflect greater hepatic clearance of toxic parent compound in the human hepatocyte coculture, which means that it requires a higher concentration of parent compound to produce an equivalent level of toxicity to HepG2 cells. For troglitazone a left shift in the primary human hepatocyte coculture toxicity curve as compared to the HepG2 cells was observed. This is thought to be due to greater generation of toxic metabolite in the primary hepatocyte coculture system and it requires lower parent concentration to produce equivalent level of toxicity.

### Example 5: Detection of Multiple Dose Dependent Toxicity

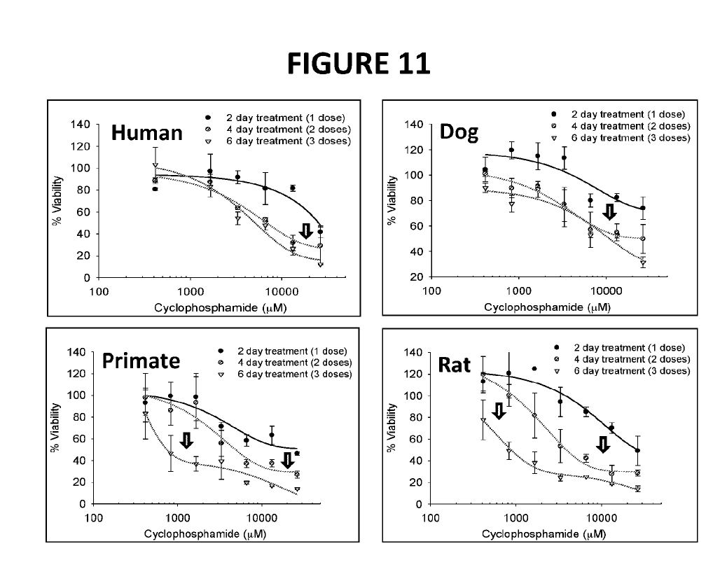

The graphs in FIGS. 11-13 present concentration dependent toxicity curves generated after 6 days of repeat compound exposure in human, dog, primate, and rat. Each compound was dosed every two days for treatment periods of two days, four days, and six days across a full range of concentrations. On the sixth day a Promega Celltiter blue assay was used to measure mitochondrial metabolic activity. That data was then used to generate a TC₅₀ value. The data demonstrate that within each species there are concentration dependent changes in toxicity over time. In the case of cyclophosphamide this is believed to occur due to the formation of a toxic metabolite. The data also show that different species produce different toxicity profiles. The data reported in FIGS. 11-13 demonstrates use of the hepatocyte-stromal cell cocultures described in Example 1 to analyze compound toxicity over an extended time period. In FIG. 11 cyclophosphamide was administered every two days for treatment periods of two days, four days, and six days across a full range of concentrations. The data clearly demonstrates that multiple dosing of the drug product shifts the toxicity curves such that slower forming toxicities may be observed. This is something that cannot be done in other systems known in the art. Among other things this data demonstrates that the systems and methods of this invention are able to detect and demonstrate interspecies differences in tox profiles.

FIG. 11 shows the hepatotoxic effect of multiple dosing of cyclophosphamide on hepatocyte-stromal cell cocultures comprising human, dog, primate, or rat primary hepatocytes. Cell viability was analyzed using a CellTiter-Blue (Promega) assay, n=4. The data show that extending the duration of dosing increases the cyclophosphamide cytotoxicity.

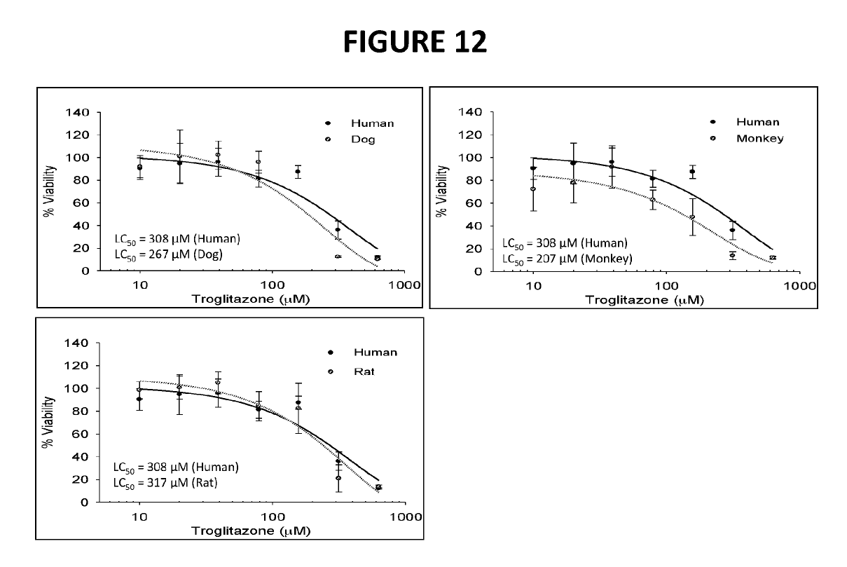

FIG. 12 shows the hepatotoxic effect (calculated LC₅₀) of multiple dosing of troglitazone on hepatocyte-stromal cell cocultures comprising human, dog, primate, or rat primary hepatocytes following six days of treatment (three doses). The results in dog, monkey, and rat, are each individually compared to human in the three graphs presented in FIG. 12.

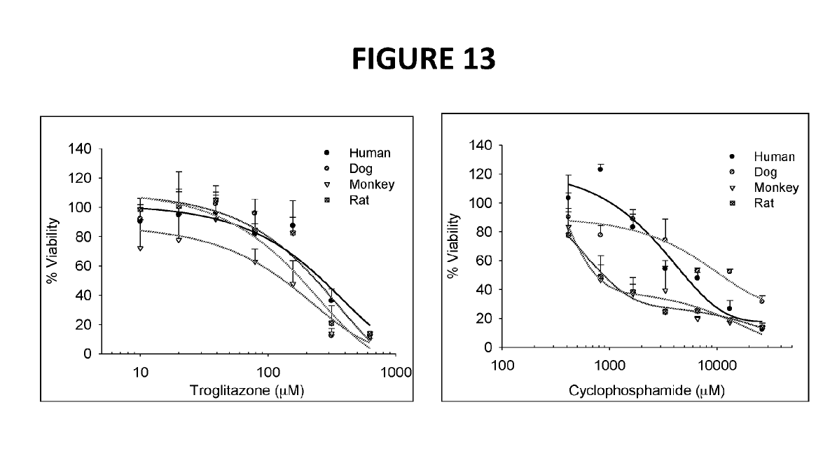

FIG. 13 summarizes the results for cyclophosphamide and troglitazone.

### Example 6: Comparison of Human, Dog, Monkey, and Rat Primary Hepatocyte-Stromal Cell Cocultures

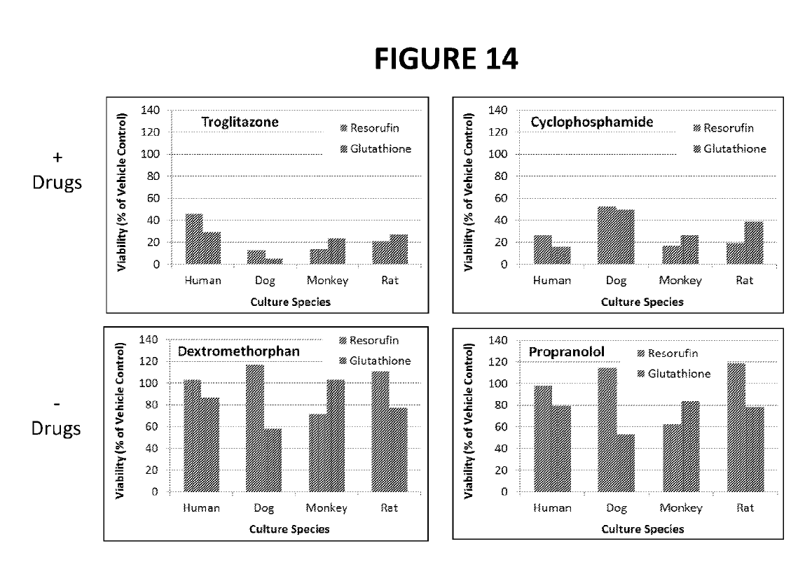

FIG. 14 presents a comparison of the GSH and Promega Celltiter blue assays. The assay was done at 50*Cmax. Each compound was dosed every two days starting on day 0. On the sixth day a Promega Celltiter blue assay to assess mitochondrial function. The very low inter-assay variability is a further demonstration of the usefulness and capabilities of the systems and methods of this disclosure.

### Example 7: A Time-Based Hepatotoxicity Assay

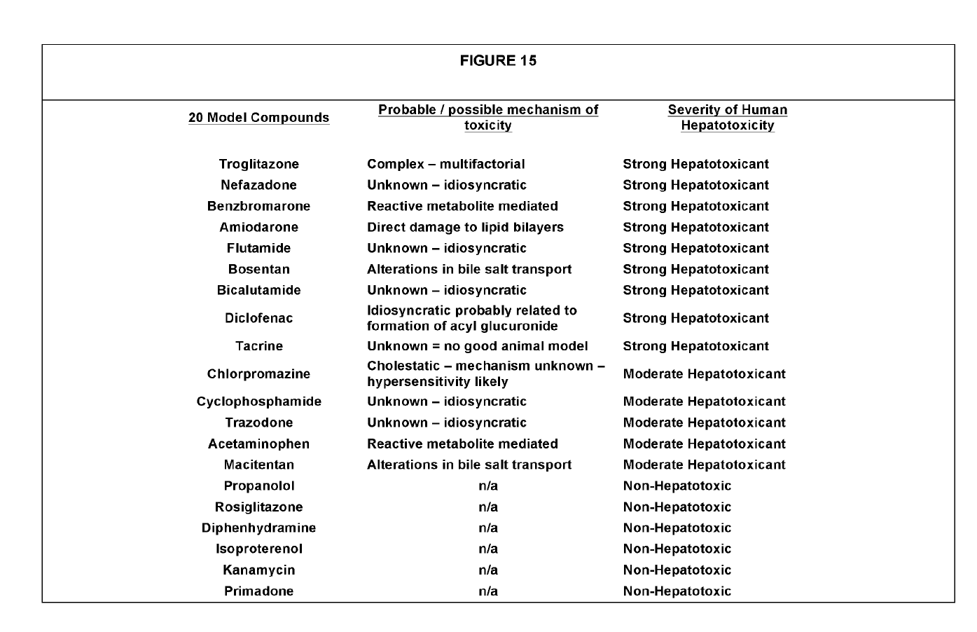

The stable, long enduring, high functionality of the primary hepatocyte-stromal cell cocultures (see Examples 1-5) enables analysis of hepatotoxicity in culture over an extended period. A panel of twenty compounds with known in vivo hepatotoxicity profiles was analyzed to evaluate the ability of this system to predict in vivo hepatotoxicity. The panel included compounds known to exhibit strong in vivo hepatotoxicity or moderate in vivo hepatotoxicity, as well as compounds that are not considered hepatotoxic (FIG. 15). The compounds that were known as hepatotoxic included compounds understood to exhibit hepatotoxicity that is (1) complex and multifactoral, (2) unknown and/or idiosyncratic, (3) ractive metabolite mediated, (4) mediated by alterations in bile salt transport, (5) and cholestatic.

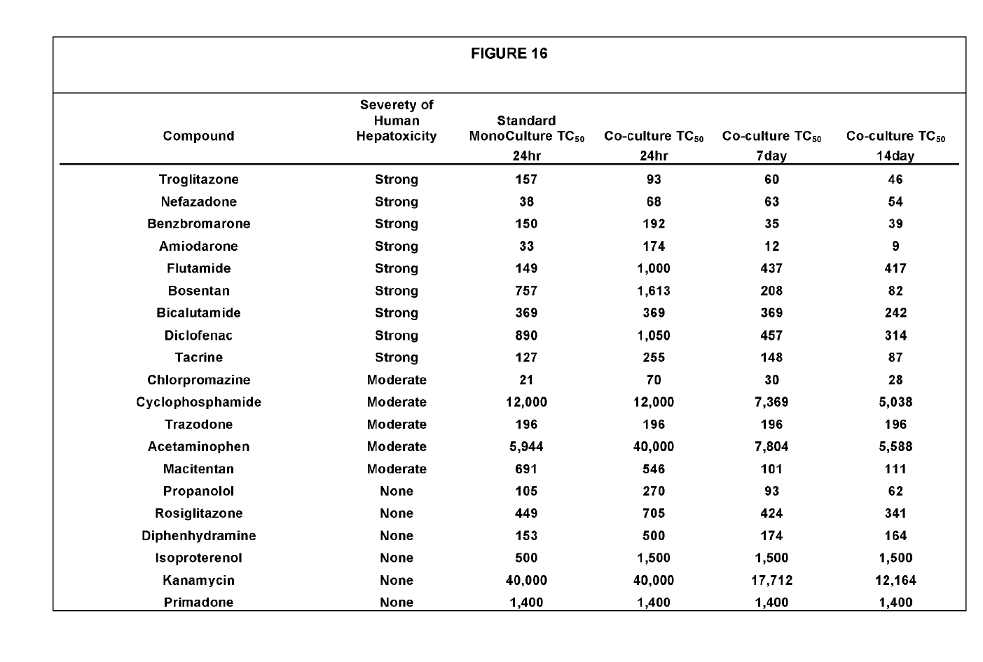

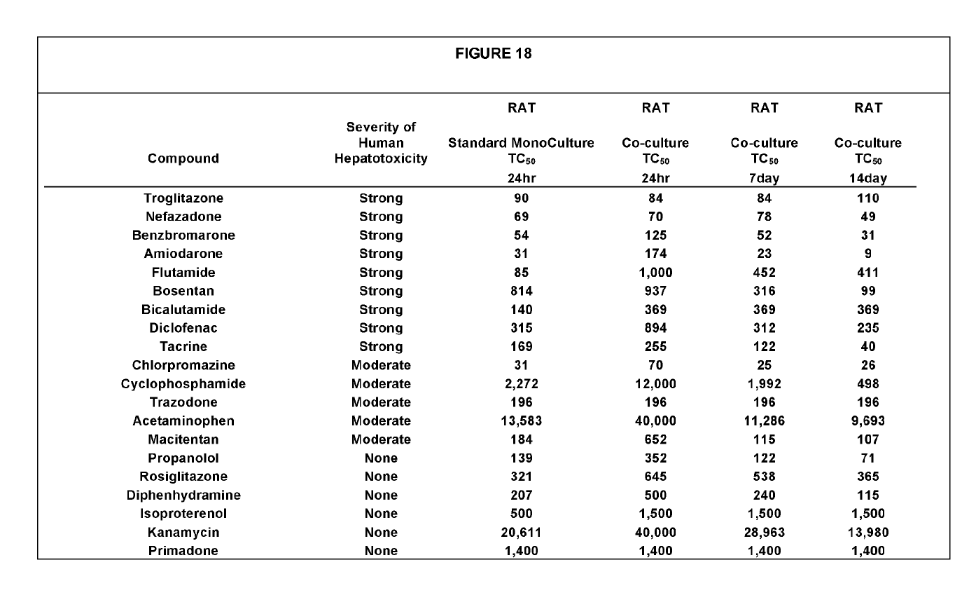

FIGS. 16 and 18 present the results when the above-elaborated twenty compounds were exposed to hepatocyte-stromal co-cultures comprising, respectively, hepatocytes from the human (FIG. 16) and from the rat (FIG. 18) species.

Human cells in “Standard Monoculture” were cryopreserved human hepatocytes that were thawed and plated in 96 well plates. They were allowed to adhere for 24 hours and then exposed to compound for 24 hours. Rat cells in “Standard Monoculture” were freshly isolated cells plated in 96 well plates. The cells were allowed to adhere for 24 hours and then exposed to compound for 24 hours. All of the co-culture were prepared according to Example 1. Compound dosing began on day 7 after seeding and fresh compound was added every 48 hours.

The data presented in FIGS. 16 and 18 shows that analyzing toxicity at a single timepoint is not a robust predictor of in vivo hepatotoxicity in this panel of compounds. For example, the human data in FIG. 16 shows that at 24 hours the LC50 of chlorpromazine is 21 and the LC50 of propranolol is 105. Those compounds are known to exhibit moderate hepatotoxicity and a lack of hepatotoxicity in vivo, respectively. In contrast, the LC50 of bosentan is 757, which considered in isolation would suggest that bosentan is a much less potent hepatotoxin than either of chlorpromazine and propranolol. However, bosentan is in fact known to exhibit strong in vivo hepatotoxicity. Similarly, the data at 7 and 14 days shows that several compounds that are not hepatotoxic in vivo or that exhibit moderate in vivo hepatotoxicity have LC50 values lower than several of the LC50 values for compounds that are known to exhibit strong hepatotoxicity in vivo. The rat data presented in FIG. 18 exhibits a similar lack of strong association between the observed in vitro toxicity and the known in vivo toxicity.

More broadly, it is widely considered by practitioners skilled in the relevant art that measurements of toxicity made at a single point in time, such as at 24 hours, hold little predictive utility and are not accorded much credibility about whether or not to progress compounds into later stages of pre-clinical or clinical drug development. For example, a test compound that produced a fairly low TC50 value of, say 100 or even 50 micromolar might be disregarded and the compound advanced onward in pre-clinical or into clinical development, with all the attendant investment of time and money that that decision to advance the compound entailed, even though that “positive liver signal” had been obtained, because the drug developers hoped or believed that the single time-point in vitro test would prove not to be predictive, or “translational,” when the drug was subsequently tested in pre-clinical animal species or in humans. Moreover, at the stage of pre-clinical development wherein they would be needed, there are no external standards against which single time point measurements of toxicity can be evaluated. One such standard, the size of the “therapeutic window,” or relationship of the maximum plasma concentration attained by a drug compared to its TC50 (i.e, TC50/Cmax), is not available when needed because the pre-clinical testing is undertaken at a stage before when the regulatory authorities, such as the U.S. Food and Drug Administration (FDA), have awarded Investigative New Drug status to the test compound. Thus it is not permitted to be administered to humans, and there is no possibility to obtain experimental Cmax values with which to compute and estimate the therapeutic window. Relatedly, for the toxicological testing of environmental and industrial chemicals, which are never deliberately administered as drugs, a Cmax value is never obtained.

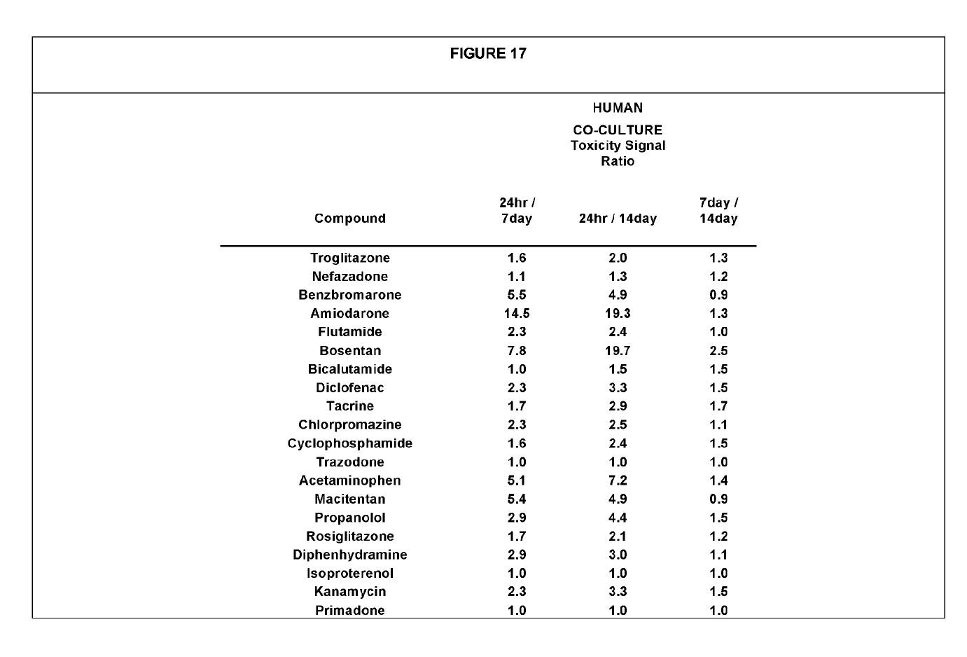

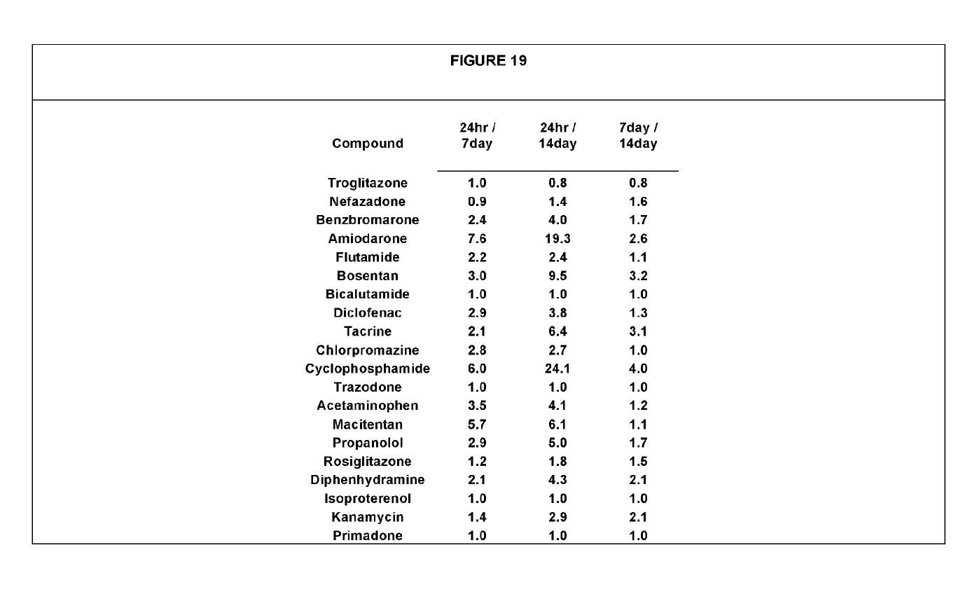

Surprisingly, in analyzing the data in FIGS. 16 and 18, it was observed that in certain instances elaborated in the following paragraphs, the ratio of hepatotoxicity at 24 h to hepatotoxicity at 7 d, or the ratio of hepatotoxicity at 24 h to hepatotoxicity at 14 d (hereinafter referred to respectively as the “24/7 toxicity ratio signal” or the 24/14 toxicity ratio signal” or, all-inclusively, as a “toxicity ratio signal”) is a useful predictor of in vivo hepatotoxicity. FIG. 17 compares the hepatotoxicity at 24 h to hepatotoxicity at 7 d, hepatotoxicity at 24 h to hepatotoxicity at 14 d, and hepatotoxicity at 7 d to hepatotoxicity at 14 d. The toxicity ratio signals are formed from the raw LC50 values presented in FIG. 16 and thus have no units. A higher LC50 value indicates a lower hepatotoxic potency and a lower LC50 value indicates a higher hepatotoxic potency. Therefore, a higher toxicity ratio signal in FIG. 17 indicates that hepatotoxicity at the later timepoint is greater than hepatotoxicity at the earlier timepoint, and a lower ratio in FIG. 17 indicates that hepatotoxicity at the later timepoint is less than hepatotoxicity at the earlier timepoint. A similar analysis of the rat data shown in FIG. 18 is presented in FIG. 19.

Based on these data a ratio of 4 was chosen as identifying a meaningfully elevated toxicity ratio signal, thus indicating that a test compound is likely to be in vivo hepatotoxic if the signal is at 4 or higher. The challenge in choosing a value for the hepatotoxicity threshold is that for the toxicity ratio signal to be experimentally valuable it must generate a very low incidence of false positive signals and therefore must not be set too low; and yet if it is set too high it may begin to miss true positive outcomes and generate signals that subsequently are found to be false negatives. Thus, the toxicity ratio signal cutoff value may be chosen in a way that incorporates a useful tradeoff between what are known in the art as sensitivity of prediction (high incidence of true negative signals) and specificity of prediction (high incidence of true positive signals). In practice, a threshold for balancing sensitivity and specificity will often be determined empirically.

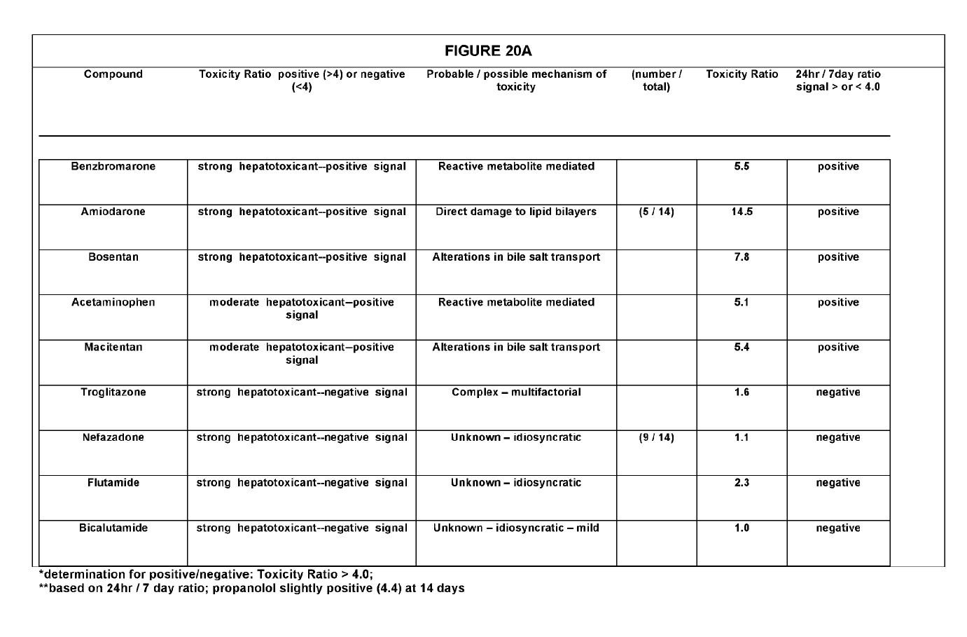

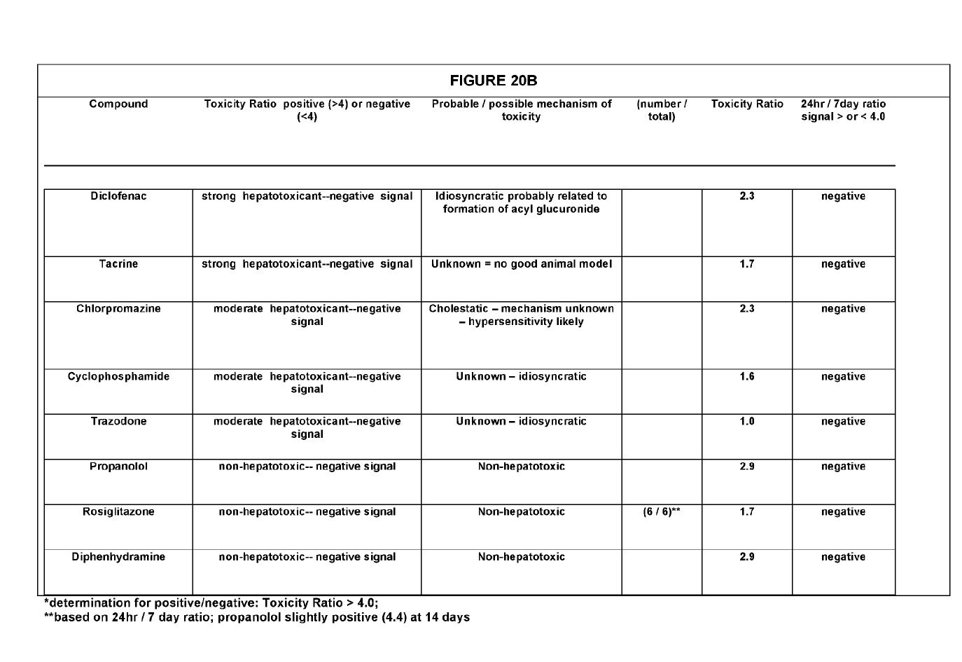

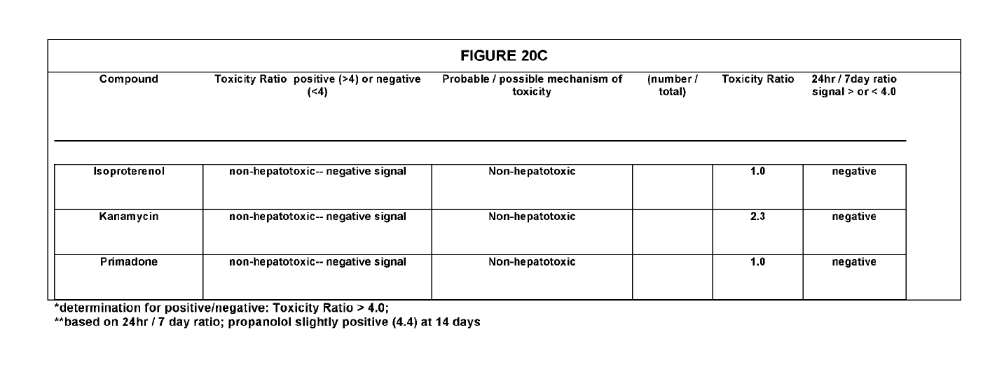

By applying a toxicity ratio cut off of 4 (values of 4 or higher indicate an actionable likelihood of in vivo hepatotoxicity and values less than or equal to 4 do not) the twenty compounds were segregated into two groups as shown in FIGS. 20A to 20C. This scoring system predicts toxicity in five of the fourteen compounds with a known in vivo hepatotoxicity and does not predict toxicity in the remaining nine. The scoring system correctly did not predict hepatotoxicity in any of the six compounds that do not exhibit hepatotoxicity in vivo.

Significantly, the five compounds scored as positive all exhibit toxicity characterized by direct damage to lipid bilayers or alterations in bile salt transport, or toxicity that is reactive metabolite mediated, and 100% of the compounds so characterized were scored as positive. Moreover, the scoring produced no false positives wherein a non-hepatotoxic compound would have been scored as positive. In other words, with this set of compounds, the toxicity ratio signal model, utilizing a hepatotoxicity cutoff score of 4, yielded a specificity (incidence of true negatives) of 100% (i.e., no false positives). Over-all, the model's sensitivity (incidence of true positives), as evaluated on the sample set of 20 reference compounds, was roughly 36%. However, in terms of the subset of the sample wherein the probable or possible mechanism of toxicity was either reactive metabolite mediated or involved alterations to bile salt transport or direct damage to the hepatocytes' lipid bilayers, the incidence of the model's generation of true positive results (sensitivity) was 100%. The foregoing mechanisms are all dependent on and correlated to the high, stable and long-enduring metabolic competency of the underlying cell-based model (in this case, the co-culture of hepatocytes and stromal cells). In contrast, the nine known hepatotoxic compounds scored as negative exhibit toxicity that is idiosyncratic or not well understood. This result is novel because in earlier days, when cell-based models possessing the innate metabolic competency of the hepatocyte-stromal cell co-culture did not exist, there were no models capable of biochemically generating the conditions that yielded the positive toxicity ratio signals disclosed herein. And the result is unexpected because until the experiments disclosed herein were conducted, there was no empirical basis for predicting that an in vitro model could reliably, with both high sensitivity and high specificity, identify the subset of hepatotoxicities that are either reactive metabolite-mediated or attributable to alterations in bile sale transport.

In short, the toxicity ratio signal is hereby shown to demonstrate excellent sensitivity (100% true negatives); and also to demonstrate excellent specificity (100% true positives) when applied to the subset of compounds wherein a reactive metabolite, alteration to bile salt transport, or damage to lipid bi-layers is implicated in the mechanism of hepatotoxic action. This finding is applicable to a substantial fraction of all chemical and/or molecular entities, and that constitutes a major leap forward in developing effective tools for pre-clinical drug development and the in vitro testing of environmental and industrial chemicals. No other currently available cell-based, in vitro method provides data that is equivalently actionable for go/no-go decision-making. Thus, the methods and systems disclosed herein demonstrate an important new tool to aid in identification of compounds for further investigation and/or development and to materially reduce the time and costs of identifying drug candidates and of drug development and, most pointedly, to reduce late-stage pre-clinical and Phase I attrition. Moreover, while the data presented herein comprises data generated with pharmaceutical test compounds, time-based toxicity ratio signals will have equally great utility in evaluating safety risks of environmental and industrial chemical entities, where metabolically responsive in vitro tools are similarly sorely lacking.

### Example 8: Prediction of Clinical and Preclinical Liver Toxicity

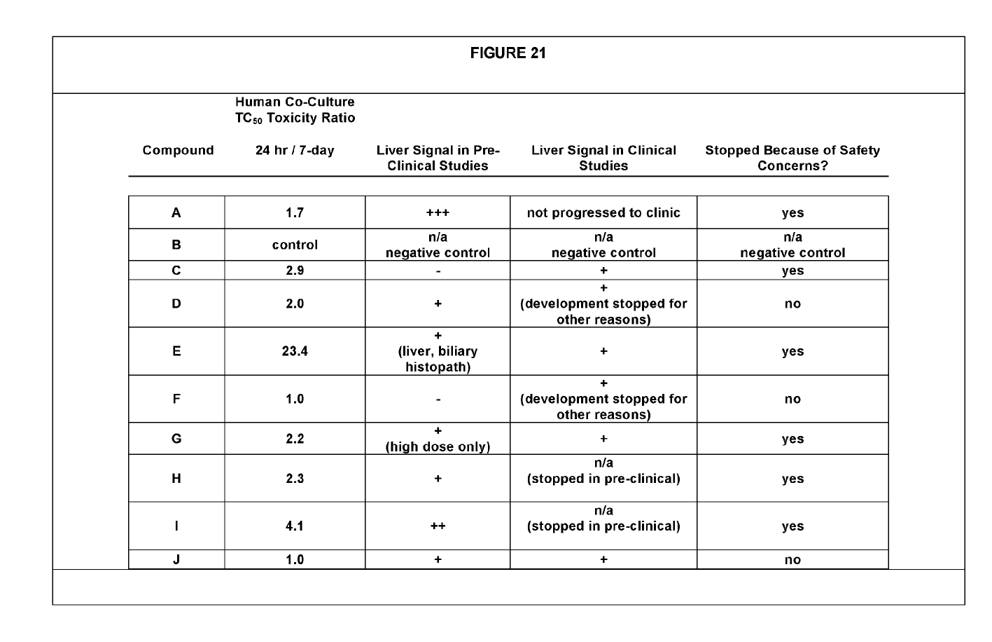

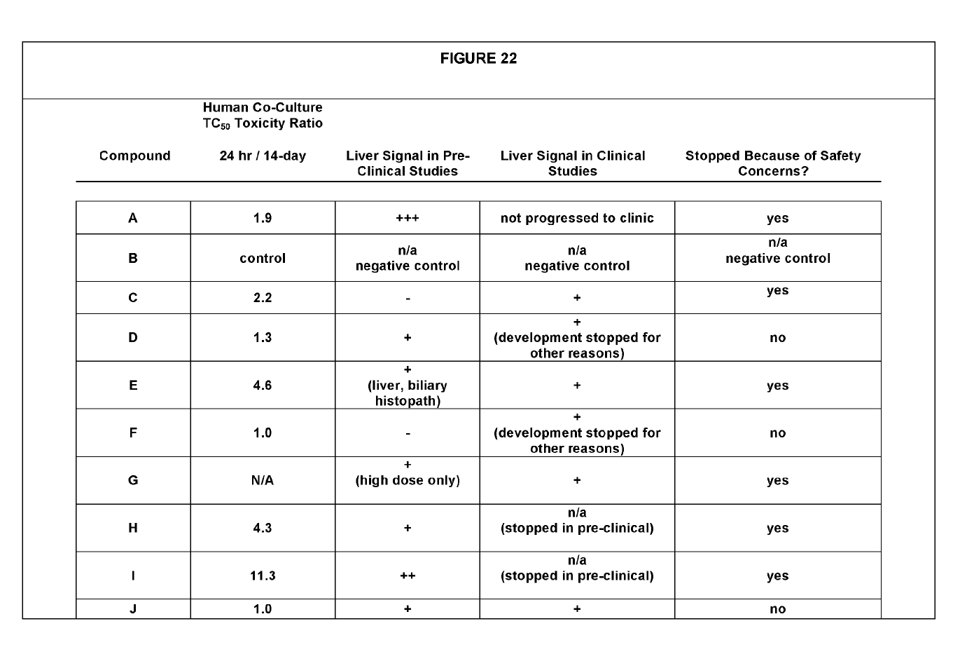

The compounds analyzed in Example 6 are all well-studied, public domain reference compounds with known hepatotoxicity profiles. To challenge the utility of the system and method in a blinded, retrospective study, the toxicity ratio signal model was applied to a set of ten pharmaceutical candidate compounds, of which one was a known negative control (i.e, known non-hepatotoxic) and nine had been discontinued from continuing pharmaceutical development either during the pre-clinical discovery stage or during Phase I clinical trials. Of the nine compounds in the test set, five were discontinued either pre-clinically or in the clinic for liver signal-related safety (i.e., hepatotoxicity) reasons, while four were discontinued for reasons that were not safety-related. As shown in FIG. 21, using the 24 h/7 d ratio and a cut off of 4, two out of five (40%) of the compounds discontinued “liver signal” for safety reasons were correctly identified by the toxicity ratio signal (compounds E and I). As shown in FIG. 22, using the 24 h/14 d ratio and a cut off of 4, three out of five (60%) of the compounds discontinued for “liver signal” safety reasons were correctly identified by the toxicity ratio signal (compounds E, H, and I). There were no false positives, in that in the instances of both the 24/7 signal and the 24/14 signal, the toxicity ratio signal did not identify as positive either the negative control compound “B” or any of the compounds that were discontinued for reasons that were not safety-related.

This data demonstrates that employing the time-based hepatotoxicity test as a component of early in vitro screening of compounds can reduce safety-related attrition in the late stages of pre-clinical drug discovery and in Phase I clinical drug development, thereby contributing materially to reduced time and invested costs in drug development, while making similar improvements to the in vitro toxicological testing of environmental and industrial chemicals.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of characterizing the time-based hepatotoxicity of a test compound, comprising:
a) incubating a first in vitro culture comprising hepatocytes with a test compound for a first culture period;
b) measuring at least one cytotoxic effect of the test compound on the hepatocytes of the first in vitro culture over the first culture period to thereby define the hepatotoxicity of the test compound over the first culture period;
c) incubating a second in vitro culture comprising hepatocytes with the test compound for a second culture period that is longer than the first culture period;
d) measuring at least one cytotoxic effect of the test compound on the hepatocytes of the second in vitro culture over the second culture period to thereby define the hepatotoxicity of the test compound over the second culture period; and
e) comparing the hepatotoxicity of the test compound over the first culture period to the hepatotoxicity of the test compound over the second culture period to thereby characterize the time-based hepatotoxicity of the test compound.

2. The method of claim 1, wherein the hepatotoxicity of the test compound over the second culture period is greater than the hepatotoxicity of the test compound over the first culture period and the test compound is identified as exhibiting time-based hepatotoxicity.

3. The method of claim 1, wherein the hepatotoxicity of the test compound over the second culture period is greater than the hepatotoxicity of the test compound over the first culture period by at least a pre-defined threshold and the test compound is identified as exhibiting time-based hepatotoxicity.

4. The method of claim 1, wherein the hepatotoxicity of the test compound over the second culture period is not greater than the hepatotoxicity of the test compound over the first culture period by at least a pre-defined threshold and the test compound is identified as not exhibiting time-based hepatotoxicity.

5. The method of claim 1, wherein the first and second in vitro cultures comprise isolated hepatocytes.

6. The method of claim 5, wherein the isolated hepatocytes are substantially dispersed across the surface of a solid substrate.

7. The method of claim 5, wherein the hepatocytes are primary hepatocytes.

8. The method of claim 5, wherein the first and second in vitro cultures further comprise at least one defined stromal cell type.

9. The method of claim 1, wherein the first culture period is from twelve hours to two days.

10. The method of claim 1, wherein the first culture period is one day.

11. The method of claim 1, wherein the second culture period is from three to twenty-eight days.

12. The method of claim 1, wherein the second culture period is from seven to fourteen days.

13. The method of claim 1, wherein the first culture period is one day and the second culture period is seven days or fourteen days.

14. The method of claim 1, wherein step a) comprises incubating a plurality of first in vitro cultures comprising hepatocytes are incubated with the test compound for the first culture period.

15. The method of claim 1, wherein step c) comprises incubating a plurality of second in vitro cultures comprising hepatocytes with the test compound for the second culture period.

16. The method of claim 1, wherein step b) comprises determining an LC₅₀ of the test compound over the first culture period.

17. The method of claim 1, wherein step d) comprises determining an LC₅₀ of the test compound over the second culture period.

18. The method of claim 1, wherein step b) comprises determining an LC₅₀ of the test compound over the first culture period, wherein step d) comprises determining an LC₅₀ of the test compound over the second culture period, and wherein step e) comprises determining the ratio of the LC₅₀ of the test compound over the first culture period to the LC₅₀ of the test compound over the second culture period to define a time based toxicity score for the test compound.
